SEQUENCE LISTING (59 sequences)

SEQ ID NO: 1 - RNA, 21 nt, synthetic construct
  source 1..21: mol_type = other RNA
ttcgcggaaggtcagcttgtg

SEQ ID NO: 2 - RNA, 21 nt, synthetic construct
  source 1..21: mol_type = other RNA
gacggcgacttgggtggagta

SEQ ID NO: 3 - RNA, 21 nt, synthetic construct
  source 1..21: mol_type = other RNA
tgtcgatgctgccggtggagg

SEQ ID NO: 4 - RNA, 21 nt, synthetic construct
  source 1..21: mol_type = other RNA
tttcgactggactctgtcctt

SEQ ID NO: 5 - RNA, 21 nt, synthetic construct
  source 1..21: mol_type = other RNA
agtacggaccactgccacctt

SEQ ID NO: 6 - RNA, 21 nt, synthetic construct
  source 1..21: mol_type = other RNA
agccgatcttggacttgacat

SEQ ID NO: 7 - RNA, 21 nt, synthetic construct
  source 1..21: mol_type = other RNA
gtacgtcccagcgtgatcttc

SEQ ID NO: 8 - RNA, 21 nt, synthetic construct
  source 1..21: mol_type = other RNA
atgtcgatgctgccggtggag

SEQ ID NO: 9 - RNA, 21 nt, synthetic construct
  source 1..21: mol_type = other RNA
ttcgactggactctgtccttg

SEQ ID NO: 10 - RNA, 21 nt, synthetic construct
  source 1..21: mol_type = other RNA
ggcgacttgggtggagtacgg

SEQ ID NO: 11 - RNA, 21 nt, synthetic construct
  source 1..21: mol_type = other RNA
ggcgacttgtacacgatctcc

SEQ ID NO: 12 - RNA, 21 nt, synthetic construct
  source 1..21: mol_type = other RNA
tatgtcgatgctgccggtgga

SEQ ID NO: 13 - RNA, 21 nt, synthetic construct
  source 1..21: mol_type = other RNA
tatgcgagcttgggtcacgtg

SEQ ID NO: 14 - RNA, 21 nt, synthetic construct
  source 1..21: mol_type = other RNA
tgtacgtcccagcgtgatctt

SEQ ID NO: 15 - RNA, 21 nt, synthetic construct
  source 1..21: mol_type = other RNA
gtcgatgctgccggtggagga

SEQ ID NO: 16 - RNA, 21 nt, synthetic construct
  source 1..21: mol_type = other RNA
aacccgtacgtcccagcgtga

SEQ ID NO: 17 - RNA, 21 nt, synthetic construct
  source 1..21: mol_type = other RNA
gtacggaccactgccaccttc

SEQ ID NO: 18 - RNA, 21 nt, synthetic construct
  source 1..21: mol_type = other RNA
gtagccgctgcgatcccctga

SEQ ID NO: 19 - RNA, 21 nt, synthetic construct
  source 1..21: mol_type = other RNA
tggcgatcttcgttttaccat

SEQ ID NO: 20 - RNA, 21 nt, synthetic construct
  source 1..21: mol_type = other RNA
ttcgtcagctagcgtggcgag

SEQ ID NO: 21 - RNA, 21 nt, synthetic construct
  source 1..21: mol_type = other RNA
tctttgcttttactgaccatg

SEQ ID NO: 22 - RNA, 20 nt, synthetic construct
  source 1..20: mol_type = other RNA
tcaagcttctcagattttac

SEQ ID NO: 23 - RNA, 19 nt, synthetic construct
  source 1..19: mol_type = other RNA
cacaagctccttccgcgag

SEQ ID NO: 24 - RNA, 19 nt, synthetic construct
  source 1..19: mol_type = other RNA
tactccacaagtcgccgtt

SEQ ID NO: 25 - RNA, 19 nt, synthetic construct
  source 1..19: mol_type = other RNA
cctccacccagcatcgata

SEQ ID NO: 26 - RNA, 19 nt, synthetic construct
  source 1..19: mol_type = other RNA
aaggacagtccagtcgaag

SEQ ID NO: 27 - RNA, 19 nt, synthetic construct
  source 1..19: mol_type = other RNA
aaggtggctggtccgtatt

SEQ ID NO: 28 - RNA, 19 nt, synthetic construct
  source 1..19: mol_type = other RNA
atgtcaagcaagatcggtt

SEQ ID NO: 29 - RNA, 19 nt, synthetic construct
  source 1..19: mol_type = other RNA
gaagatcactgggacgtat

SEQ ID NO: 30 - RNA, 19 nt, synthetic construct
  source 1..19: mol_type = other RNA
ctccaccgagcatcgatat

SEQ ID NO: 31 - RNA, 19 nt, synthetic construct
  source 1..19: mol_type = other RNA
caaggacagtccagtcgaa

SEQ ID NO: 32 - RNA, 19 nt, synthetic construct
  source 1..19: mol_type = other RNA
ccgtactccccaagtcgtt

SEQ ID NO: 33 - RNA, 19 nt, synthetic construct
  source 1..19: mol_type = other RNA
ggagatcgtacaagtcgtt

SEQ ID NO: 34 - RNA, 19 nt, synthetic construct
  source 1..19: mol_type = other RNA
tccaccgggcatcgacatg

SEQ ID NO: 35 - RNA, 19 nt, synthetic construct
  source 1..19: mol_type = other RNA
cacgtgacaagctcgcatg

SEQ ID NO: 36 - RNA, 19 nt, synthetic construct
  source 1..19: mol_type = other RNA
aagatcactgggacgtatg

SEQ ID NO: 37 - RNA, 19 nt, synthetic construct
  source 1..19: mol_type = other RNA
tcctccacgcagcatcgat

SEQ ID NO: 38 - RNA, 19 nt, synthetic construct
  source 1..19: mol_type = other RNA
tcacgctgacgtacgggtt

SEQ ID NO: 39 - RNA, 19 nt, synthetic construct
  source 1..19: mol_type = other RNA
gaaggtgggtggtccgtat

SEQ ID NO: 40 - RNA, 19 nt, synthetic construct
  source 1..19: mol_type = other RNA
tcaggggagcagcggctat

SEQ ID NO: 41 - RNA, 19 nt, synthetic construct
  source 1..19: mol_type = other RNA
atggtaaagaagatcgtta

SEQ ID NO: 42 - RNA, 19 nt, synthetic construct
  source 1..19: mol_type = other RNA
ctcgccactagctgacgag

SEQ ID NO: 43 - RNA, 19 nt, synthetic construct
  source 1..19: mol_type = other RNA
catggtcaaaaagcaaaga

SEQ ID NO: 44 - RNA, 18 nt, synthetic construct
  source 1..18: mol_type = other RNA
gtaaaatcagaagcttga

SEQ ID NO: 45 - DNA, 19 bp, synthetic construct
  source 1..19: mol_type = other DNA
gttttggccactgactgac

SEQ ID NO: 46 - RNA, 19 nt, synthetic construct
  source 1..19: mol_type = other RNA
gttttggccactgactgac

SEQ ID NO: 47 - DNA, 72 bp, synthetic construct
  source 1..72: mol_type = other DNA
ctggaggcttgctgaaggctgtatgctgcaggacacaaggcctgttactagcactcacatggaacaaatggc

SEQ ID NO: 48 - DNA, 128 bp, synthetic construct
  source 1..128: mol_type = other DNA
ctggaggcttgctgaaggctgtatgctgtacgatctaatatcgctcgttttggccactgactgacgagcgatatgatcgtacgacaggacacaaggcctgttactagcactcacatggaacaaatggc

SEQ ID NO: 49 - DNA, 78 bp, synthetic construct
  source 1..78: mol_type = other DNA
cactccctctctgcgcgctcgctcgctcactgaggccgggcgaccaaaggtcgcccacgcccgggctttgcccgggcg

SEQ ID NO: 50 - DNA, 145 bp, synthetic construct
  source 1..145: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 51 - RNA, 21 nt, synthetic construct
  source 1..21: mol_type = other RNA
  misc_difference 1: note = n is C, A or G
ntcgcggaaggtcagcttgtg

SEQ ID NO: 52 - RNA, 21 nt, synthetic construct
  source 1..21: mol_type = other RNA
  misc_difference 1: note = n is C, A or G
nttcgactggactctgtcctt

SEQ ID NO: 53 - RNA, 21 nt, synthetic construct
  source 1..21: mol_type = other RNA
  misc_difference 1: note = n is C, A or G
natgtcgatgctgccggtgga

SEQ ID NO: 54 - RNA, 21 nt, synthetic construct
  source 1..21: mol_type = other RNA
  misc_difference 1: note = n is C, A or G
natgcgagcttgggtcacgtg

SEQ ID NO: 55 - RNA, 21 nt, synthetic construct
  source 1..21: mol_type = other RNA
  misc_difference 1: note = n is C, A or G
ngtacgtcccagcgtgatctt

SEQ ID NO: 56 - RNA, 21 nt, synthetic construct
  source 1..21: mol_type = other RNA
  misc_difference 1: note = n is C, A or G
ntcgtcagctagcgtggcgag

SEQ ID NO: 57 - protein, 7 aa, synthetic construct
KGGGFHG

SEQ ID NO: 58 - protein, 12 aa, synthetic construct
AAAKGGGFHGAS

SEQ ID NO: 59 - protein, 748 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAAAKGGGFHGASAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

(19) United States
(12) Patent Application Publication
(10) Pub. No.: US 2025/0197862 A1
(43) Pub. Date: Jun. 19, 2025

(54) ARTIFICIAL microRNAs TARGETING TAU

(71) Applicant: Genzyme Corporation, Cambridge, MA (US)

(72) Inventors: Bradford Miller ELMER, Arlington, MA (US); Jessica Marie HOGESTYN, Framingham, MA (US); Shyam RAMACHANDRAN, Winchester, MA (US); Brenda RICHARDS, Hopkinton, MA (US)

(21) Appl. No.: 18/978,680

(22) Filed: Dec. 12, 2024

Related U.S. Application Data
(60) Provisional application No. 63/610,638, filed on Dec. 15, 2023.

(51) Int. Cl. C12N 15/113; A61P 25/28; C12N 15/86

(52) U.S. Cl. CPC C12N 15/113; A61P 25/28; C12N 15/86; C12N 2310/141; C12N 2750/14122; C12N 2750/14143

(57) ABSTRACT

Provided herein are artificial microRNA (miRNA) molecules for treating tauopathies. In some embodiments, the miRNA molecules target expression of Tau protein. Further provided herein are expression constructs, vectors (e.g., rAAV), cells, viral particles, and pharmaceutical compositions containing the artificial miRNA molecules. Yet further provided herein are methods and kits related to the use of the miRNA molecules, for example, to treat tauopathies including is Alzheimer's disease, progressive supranuclear palsy, corticobasal degeneration, frontotemporal dementia with parkinsonism-17, Pick's Disease, argyrophilic grain disease, globular glial tauopathy, chronic traumatic encephalopathy and post-encephalitic parkinsonism.

### CROSS REFERENCE TO RELATED APPLICATIONS

This application claims the priority benefit of U.S. Provisional Application No. 63/610,638, filed Dec. 15, 2023, which is incorporated by reference in its entirety.

### REFERENCE TO AN ELECTRONIC SEQUENCE LISTING

The content of the electronic sequence listing (159792018300seqlist.xml; Size: 56,193 bytes; and Date of Creation: Dec. 10, 2024) is herein incorporated by reference in its entirety.

### FIELD OF THE DISCLOSURE

The present disclosure relates to artificial microRNA molecules. In some aspects, the disclosure relates to artificial microRNA molecules that reduce expression of microtubule associated protein tau.

### BACKGROUND

The accumulation of pathological tau protein drives neurotoxicity and regional brain atrophy in a group of neurodegenerative diseases known as tauopathies (e.g. Progressive upranuclear palsy, corticobasal degeneration, Alzheimer's disease). Neurodegeneration manifests with progressive and fatal cognitive and motor impairments, and there are no approved treatments known to modify disease course. The microtubule associated protein tau, is a well-credentialed therapeutic target, given that i) the strongest known genetic risk factors are those affecting the tau-encoding MAPT gene, and ii) clinical impairment significantly correlates with tau burden. Tau reduction in preclinical models not only prevents further aggregation, but can enable clearance of existing tau aggregates.

Current treatments for tauopathy are largely symptomatic and supportive, and none have been shown to significantly reduce tau pathology. While many tau-targeted therapeutics are in development, most of the therapeutics target the tau protein itself. Given the heterogeneity of tau isoform expression, posttranslational modification, and conformation between tauopathies, targeting a specific form of tau protein directly may not have the broadest therapeutic impact.

### BRIEF SUMMARY

The disclosure provides artificial microRNA (miRNA) molecules targeting human MAPT mRNA. MAPT-targeted artificial miRNA gene therapy stands to markedly improve quality of life and slow disease progression. The constructs described herein have wide commercial applicability in both rare and common neurodegenerative tauopathies.

In one aspect, the disclosure artificial miRNA comprising a first stand and a second strand, wherein
(a) the first strand and second strand form a duplex;
(b) the first strand comprises a guide region comprising a nucleotide sequence having at least about 90% identity or at least about 95% identity to the sequence of SEQ ID NO: 1 (5′-UUCGCGGAAGGUCAGCUUGUG-3′), SEQ ID NO: 2 (5′-GACGGCGACUUGGGUGGAGUA-3′), SEQ ID NO: 3 (5′-UGUCGAUGCUGCCGGUGGAGG-3′), SEQ ID NO: 4 (5′-UUUCGACUGGACUCUGUCCUU-3′), SEQ ID NO 5 (5′-AGUACGGACCACUGCCACCUU-3′), SEQ ID NO: 6 (5′-AGCCGAUCUUGGACUUGACAU-3′), SEQ ID NO: 7 (5′-GUACGUCCCAGCGUGAUCUUC-3′), SEQ ID NO: 8 (5′-AUGUCGAUGCUGCCGGUGGAG-3′), SEQ ID NO: 9 (5′-UUCGACUGGACUCUGUCCUUG-3′), SEQ ID NO: 10 (5′-GGCGACUUGGGUGGAGUACGG-3′), SEQ ID NO: 11 (5′-GGCGACUUGUACACGAUCUCC-3′), SEQ ID NO: 12 (5′-UAUGUCGAUGCUGCCGGUGGA-3′), SEQ ID NO: 13 (5′-UAUGCGAGCUUGGGUCACGUG-3′), SEQ ID NO: 14 (5′-UGUACGUCCCAGCGUGAUCUU-3′), SEQ ID NO: 15 (5′-GUCGAUGCUGCCGGUGGAGGA-3′), SEQ ID NO: 16 (5′-AACCCGUACGUCCCAGCGUGA-3′), SEQ ID NO: 17 (5′-GUACGGACCACUGCCACCUUC-3′), SEQ ID NO: 18 (5′-GUAGCCGCUGCGAUCCCCUGA-3′), SEQ ID NO: 19 (5′-UGGCGAUCUUCGUUUUACCAU-3′), SEQ ID NO: 20 (5′-UUCGUCAGCUAGCGUGGCGAG-3′), SEQ ID NO: 21 (5′-UCUUUGCUUUUACUGACCAUG-3′), or SEQ ID NO: 22 (5′-UCAAGCUUCUCAGAUUUUAC-3′); and
(c) the second strand (passenger strand) comprises a non-guide region that comprises a nucleotide sequence that is fully or partially complementary to the nucleotide sequence of the guide region.

In some embodiments, the first strand comprises a guide sequence having the sequence SEQ ID NO: 1 (5′-UUCGCGGAAGGUCAGCUUGUG-3′), SEQ ID NO: 2 (5′-GACGGCGACUUGGGUGGAGUA-3′), SEQ ID NO: 3 (5′-UGUCGAUGCUGCCGGUGGAGG-3′), SEQ ID NO: 4 (5′-UUUCGACUGGACUCUGUCCUU-3′), SEQ ID NO: 5 (5′-AGUACGGACCACUGCCACCUU-3′), SEQ ID NO: 6 (5′-AGCCGAUCUUGGACUUGACAU-3′), SEQ ID NO: 7 (5′-GUACGUCCCAGCGUGAUCUUC-3′), SEQ ID NO: 8 (5′-AUGUCGAUGCUGCCGGUGGAG-3′), SEQ ID NO: 9 (5′-UUCGACUGGACUCUGUCCUUG-3′), SEQ ID NO: 10 (5′-GGCGACUUGGGUGGAGUACGG-3′), SEQ ID NO: 11 (5′-GGCGACUUGUACACGAUCUCC-3′), SEQ ID NO: 12 (5′-UAUGUCGAUGCUGCCGGUGGA-3′), SEQ ID NO: 13 (5′-UAUGCGAGCUUGGGUCACGUG-3′), SEQ ID NO: 14 (5′-UGUACGUCCCAGCGUGAUCUU-3′), SEQ ID NO: 15 (5′-GUCGAUGCUGCCGGUGGAGGA-3′), SEQ ID NO: 16 (5′-AACCCGUACGUCCCAGCGUGA-3′), SEQ ID NO: 17 (5′-GUACGGACCACUGCCACCUUC-3′), SEQ ID NO: 18 (5′-GUAGCCGCUGCGAUCCCCUGA-3′), SEQ ID NO: 19 (5′-UGGCGAUCUUCGUUUUACCAU-3′), SEQ ID NO: 20 (5′-UUCGUCAGCUAGCGUGGCGAG-3′), SEQ ID NO: 21 (5′-UCUUUGCUUUUACUGACCAUG-3′), or SEQ ID NO: 22 (5′-UCAAGCUUCUCAGAUUUUAC-3′).

In one embodiment, the guide sequence comprises the sequence having at least about 90% identity or at least about 95% identity to the sequence of SEQ ID NO: 1 (5′-UUCGCGGAAGGUCAGCUUGUG-3′) and the non-guide region comprises the sequence having at least about 90% identity or at least about 95% identity to the sequence of SEQ ID NO: 23 (5′-CACAAGCUCCUUCCGCGAG-3′). In one such embodiment, the guide sequence comprises the sequence of SEQ ID NO: 1 (5′-UUCGCGGAAGGUCAGCUUGUG-3′) and the non-guide region comprises the sequence of SEQ ID NO: 23 (5′-CACAAGCUCCUUCCGCGAG-3′).

In another embodiment, the guide sequence comprises the sequence having at least about 90% identity or at least about 95% identity to the sequence of SEQ ID NO: 2 (5′-GACGGCGACUUGGGUGGAGUA-3′) and the non-guide region comprises the sequence having at least about 90% identity or at least about 95% identity to the sequence of SEQ ID NO: 24 (5′-UACUCCACAAGUCGCCGUU-3′). In one such embodiment, the guide sequence comprises the sequence of SEQ ID NO: 2 (5′-GACGGCGACUUGGGUGGAGUA-3′) and the non-guide region comprises the sequence of SEQ ID NO: 24 (5′-UACUCCACAAGUCGCCGUU-3′).

In another embodiment, the guide sequence comprises the sequence having at least about 90% identity or at least about 95% identity to the sequence of SEQ ID NO: 3 (5′-UGUCGAUGCUGCCGGUGGAGG-3′) and the non-guide region comprises the sequence having at least about 90% identity or at least about 95% identity to the sequence of SEQ ID NO: 25 (5′-CCUCCACCCAGCAUCGAUA-3′). In one such embodiment, the guide sequence comprises the sequence of SEQ ID NO: 3 (5′-UGUCGAUGCUGCCGGUGGAGG-3′) and the non-guide region comprises the sequence of SEQ ID NO: 25 (5′-CCUCCACCCAGCAUCGAUA-3′).

In another embodiment, the guide sequence comprises the sequence having at least about 90% identity or at least about 95% identity to the sequence of SEQ ID NO: 4 (5′-UUUCGACUGGACUCUGUCCUU-3′) and the non-guide region comprises the sequence having at least about 90% identity or at least about 95% identity to the sequence of SEQ ID NO: 26 (5′-AAGGACAGUCCAGUCGAAG-3′). In one such embodiment, the guide sequence comprises the sequence of SEQ ID NO: 4 (5′-UUUCGACUGGACUCUGUCCUU-3′) and the non-guide region comprises the sequence of SEQ ID NO: 26 (5′-AAGGACAGUCCAGUCGAAG-3′).

In another embodiment, the guide sequence comprises the sequence having at least about 90% identity or at least about 95% identity to the sequence of SEQ ID NO: 5 (5′-AGUACGGACCACUGCCACCUU-3′) and the non-guide region comprises the sequence having at least about 90% identity or at least about 95% identity to the sequence of SEQ ID NO: 27 (5′-AAGGUGGCUGGUCCGUAUU-3′). In one such embodiment, the guide sequence comprises the sequence of SEQ ID NO: 5 (5′-AGUACGGACCACUGCCACCUU-3′) and the non-guide region comprises the sequence of SEQ ID NO: 27 (5′-AAGGUGGCUGGUCCGUAUU-3′).

In another embodiment, the guide sequence comprises the sequence having at least about 90% identity or at least about 95% identity to the sequence of SEQ ID NO: 6 (5′-AGCCGAUCUUGGACUUGACAU-3′) and the non-guide region comprises the sequence having at least about 90% identity or at least about 95% identity to the sequence of SEQ ID NO: 28 (5′-AUGUCAAGCAAGAUCGGUU-3′). In one such embodiment, the guide sequence comprises the sequence of SEQ ID NO: 5 (5′-AGUACGGACCACUGCCACCUU-3′) and the non-guide region comprises the sequence of SEQ ID NO: 28 (5′-AAGGUGGCUGGUCCGUAUU-3′).

In another embodiment, the guide sequence comprises the sequence having at least about 90% identity or at least about 95% identity to the sequence of SEQ ID NO: 7 (5′-GUACGUCCCAGCGUGAUCUUC-3′) and the non-guide region comprises the sequence having at least about 90% identity or at least about 95% identity to the sequence of SEQ ID NO: 29 (5′-GAAGAUCACUGGGACGUAU-3′). In one such embodiment, the guide sequence comprises the sequence of SEQ ID NO: 7 (5′-GUACGUCCCAGCGUGAUCUUC-3′) and the non-guide region comprises the sequence of SEQ ID NO: 29 (5′-GAAGAUCACUGGGACGUAU-3′).

In another embodiment, the guide sequence comprises the sequence having at least about 90% identity or at least about 95% identity to the sequence of SEQ ID NO: 8 (5′-AUGUCGAUGCUGCCGGUGGAG-3′) and the non-guide region comprises the sequence having at least about 90% identity or at least about 95% identity to the sequence of SEQ ID NO: 30 (5′-CUCCACCGAGCAUCGAUAU-3′). In one such embodiment, the guide sequence comprises the sequence of SEQ ID NO: 8 (5′-AUGUCGAUGCUGCCGGUGGAG-3′) and the non-guide region comprises the sequence of SEQ ID NO: 30 (5′-CUCCACCGAGCAUCGAUAU-3′).

In another embodiment, the guide sequence comprises the sequence having at least about 90% identity or at least about 95% identity to the sequence of SEQ ID NO: 9 (5′-UUCGACUGGACUCUGUCCUUG-3′) and the non-guide region comprises the sequence having at least about 90% identity or at least about 95% identity to the sequence of SEQ ID NO: 31 (5′-CAAGGACAGUCCAGUCGAA-3′). In one such embodiment, the guide sequence comprises the sequence of SEQ ID NO: 9 (5′-AUGUCGAUGCUGCCGGUGGAG-3′) and the non-guide region comprises the sequence of SEQ ID NO: 31 (5′-CUCCACCGAGCAUCGAUAU-3′).

In another embodiment, the guide sequence comprises the sequence having at least about 90% identity or at least about 95% identity to the sequence of SEQ ID NO: 10 (5′-GGCGACUUGGGUGGAGUACGG-3′) and the non-guide region comprises the sequence having at least about 90% identity or at least about 95% identity to the sequence of SEQ ID NO: 32 (5′-CCGUACUCCCCAAGUCGUU-3′). In one such embodiment, the guide sequence comprises the sequence of SEQ ID NO: 10 (5′-GGCGACUUGGGUGGAGUACGG-3′) and the non-guide region comprises the sequence of SEQ ID NO: 32 (5′-CCGUACUCCCCAAGUCGUU-3′).

In another embodiment, the guide sequence comprises the sequence having at least about 90% identity or at least about 95% identity to the sequence of SEQ ID NO: 11 (5′-GGCGACUUGUACACGAUCUCC-3′) and the non-guide region comprises the sequence having at least about 90% identity or at least about 95% identity to the sequence of SEQ ID NO: 33 (5′-GGAGAUCGUACAAGUCGUU-3′). In one such embodiment, the guide sequence comprises the sequence of SEQ ID NO: 11 (5′-GGCGACUUGUACACGAUCUCC-3′) and the non-guide region comprises the sequence of SEQ ID NO: 33 (5′-GGAGAUCGUACAAGUCGUU-3′).

In another embodiment, the guide sequence comprises the sequence having at least about 90% identity or at least about 95% identity to the sequence of SEQ ID NO: 12 (5′-UAUGUCGAUGCUGCCGGUGGA-3′) and the non-guide region comprises the sequence having at least about 90% identity or at least about 95% identity to the sequence of SEQ ID NO: 34 (5′-UCCACCGGGCAUCGACAUG-3′). In one such embodiment, the guide sequence comprises the sequence of SEQ ID NO: 12 (5′-UAUGUCGAUGCUGCCGGUGGA-3′) and the non-guide region comprises the sequence of SEQ ID NO: 34 (5′-UCCACCGGGCAUCGACAUG-3′).

In another embodiment, the guide sequence comprises the sequence having at least about 90% identity or at least about 95% identity to the sequence of SEQ ID NO: 13 (5′-UAUGCGAGCUUGGGUCACGUG-3′) and the non-guide region comprises the sequence having at least about 90% identity or at least about 95% identity to the sequence of SEQ ID NO: 35 (5′-CACGUGACAAGCUCGCAUG-3′). In one such embodiment, the guide sequence comprises the sequence of SEQ ID NO: 13 (5′-UAUGCGAGCUUGGGUCACGUG-3′) and the non-guide region comprises the sequence of SEQ ID NO: 35 (5′-CACGUGACAAGCUCGCAUG-3′).

In another embodiment, the guide sequence comprises the sequence having at least about 90% identity or at least about 95% identity to the sequence of SEQ ID NO: 14 (5′-UGUACGUCCCAGCGUGAUCUU-3′) and the non-guide region comprises the sequence having at least about 90% identity or at least about 95% identity to the sequence of SEQ ID NO: 36 (5′-AAGAUCACUGGGACGUAUG-3′). In one such embodiment, the guide sequence comprises the sequence of SEQ ID NO: 14 (5′-UGUACGUCCCAGCGUGAUCUU-3′) and the non-guide region comprises the sequence of SEQ ID NO: 36 (5′-AAGAUCACUGGGACGUAUG-3′).

In another embodiment, the guide sequence comprises the sequence having at least about 90% identity or at least about 95% identity to the sequence of SEQ ID NO: 15 (5′-GUCGAUGCUGCCGGUGGAGGA-3′) and the non-guide region comprises the sequence having at least about 90% identity or at least about 95% identity to the sequence of SEQ ID NO: 37 (UCCUCCACGCAGCAUCGAU). In one such embodiment, the guide sequence comprises the sequence of SEQ ID NO: 15 (5′-GUCGAUGCUGCCGGUGGAGGA-3′) and the non-guide region comprises the sequence of SEQ ID NO: 37 (UCCUCCACGCAGCAUCGAU).

In another embodiment, the guide sequence comprises the sequence having at least about 90% identity or at least about 95% identity to the sequence of SEQ ID NO: 16 (5′-AACCCGUACGUCCCAGCGUGA-3′) and the non-guide region comprises the sequence having at least about 90% identity or at least about 95% identity to the sequence of SEQ ID NO: 38 (5′-UCACGCUGACGUACGGGUU-3′). In one such embodiment, the guide sequence comprises the sequence of SEQ ID NO: 16 (5′-AACCCGUACGUCCCAGCGUGA-3′) and the non-guide region comprises the sequence of SEQ ID NO: 38 (5′-UCACGCUGACGUACGGGUU-3′).

In another embodiment, the guide sequence comprises the sequence having at least about 90% identity or at least about 95% identity to the sequence of SEQ ID NO: 17 (5′-GUACGGACCACUGCCACCUUC-3′) and the non-guide region comprises the sequence having at least about 90% identity or at least about 95% identity to the sequence of SEQ ID NO: 39 (5′-GAAGGUGGGUGGUCCGUAU-3′). In one such embodiment, the guide sequence comprises the sequence of SEQ ID NO: 17 (5′-GUACGGACCACUGCCACCUUC-3′) and the non-guide region comprises the sequence of SEQ ID NO: 39 (5′-GAAGGUGGGUGGUCCGUAU-3′).

In another embodiment, the guide sequence comprises the sequence having at least about 90% identity or at least about 95% identity to the sequence of SEQ ID NO: 18 (5′-GUAGCCGCUGCGAUCCCCUGA-3′) and the non-guide region comprises the sequence having at least about 90% identity or at least about 95% identity to the sequence of SEQ ID NO: 40 (5′-UCAGGGGAGCAGCGGCUAU-3′). In one such embodiment, the guide sequence comprises the sequence of SEQ ID NO: 18 (5′-GUAGCCGCUGCGAUCCCCUGA-3′) and the non-guide region comprises the sequence of SEQ ID NO: 40 (5′-UCAGGGGAGCAGCGGCUAU-3′).

In another embodiment, the guide sequence comprises the sequence having at least about 90% identity or at least about 95% identity to the sequence of SEQ ID NO: 19 (5′-UGGCGAUCUUCGUUUUACCAU-3′) and the non-guide region comprises the sequence having at least about 90% identity or at least about 95% identity to the sequence of SEQ ID NO: 41 (5′-AUGGUAAAGAAGAUCGUUA-3′). In one such embodiment, the guide sequence comprises the sequence of SEQ ID NO: 19 (5′-UGGCGAUCUUCGUUUUACCAU-3′) and the non-guide region comprises the sequence of SEQ ID NO: 41 (5′-AUGGUAAAGAAGAUCGUUA-3′).

In another embodiment, the guide sequence comprises the sequence having at least about 90% identity or at least about 95% identity to the sequence of SEQ ID NO: 20 (5′-UUCGUCAGCUAGCGUGGCGAG-3′) and the non-guide region comprises the sequence having at least about 90% identity or at least about 95% identity to the sequence of SEQ ID NO: 42 (5′-CUCGCCACUAGCUGACGAG-3′). In one such embodiment, the guide sequence comprises the sequence of SEQ ID NO: 20 (5′-UUCGUCAGCUAGCGUGGCGAG-3′) and the non-guide region comprises the sequence of SEQ ID NO: 42 (5′-CUCGCCACUAGCUGACGAG-3′).

In another embodiment, the guide sequence comprises the sequence having at least about 90% identity or at least about 95% identity to the sequence of SEQ ID NO: 21 (5′-UCUUUGCUUUUACUGACCAUG-3′) and the non-guide region comprises the sequence having at least about 90% identity or at least about 95% identity to the sequence of SEQ ID NO: 43 (5′-CAUGGUCAAAAAGCAAAGA-3′). In one such embodiment, the guide sequence comprises the sequence of SEQ ID NO: 21 (5′-UCUUUGCUUUUACUGACCAUG-3′) and the non-guide region comprises the sequence of SEQ ID NO: 43 (5′-CAUGGUCAAAAAGCAAAGA-3′).

In another embodiment, the guide sequence comprises the sequence having at least about 90% identity or at least about 95% identity to the sequence of SEQ ID NO: 22 (5′-UCAAGCUUCUCAGAUUUUAC-3′) and the non-guide region comprises the sequence having at least about 90% identity or at least about 95% identity to the sequence of SEQ ID NO: 44 (5′-GUAAAAUCAGAAGCUUGA-3′). In one such embodiment, the guide sequence comprises the sequence of SEQ ID NO: 22 (5′-UCAAGCUUCUCAGAUUUUAC-3′) and the non-guide region comprises the sequence of SEQ ID NO: 44 (5′-GUAAAAUCAGAAGCUUGA-3′).

In any of the embodiments herein, the guide strand and the non-guide strand can be linked by means of a RNA linker capable of forming a loop structure. In some embodiments, the RNA linker comprises from 4 to 50 nucleotides. In some embodiments, the loop structure comprises 4 to 20 nucleotides.

In some embodiments of the above aspect and embodiments, the artificial miRNA molecules targets the 3′-untranslated region (3′-UTR) of tau mRNA. In some embodiments, the artificial miRNA molecules show low off-target potential.

In some embodiments of the above aspect and embodiments, the disclosure provides an expression construct comprising nucleic acid encoding the artificial miRNA molecules described herein. In some embodiments, the nucleic acid encoding the artificial miRNA molecules are embedded in a miRNA scaffold. In some embodiments, the nucleic acid encoding the artificial miRNA is operably linked to a promoter. In some embodiments, the promoter is selected from a cytomegalovirus (CMV) immediate early promoter, an RSV LTR, a MoMLV LTR, a phosphoglycerate kinase-1 (PGK) promoter, a simian virus 40 (SV40) promoter, a CK6 promoter, a transthyretin promoter (TTR), a TK promoter, a tetracycline responsive promoter (TRE), an HBV promoter, an hAAT promoter, a LSP promoter, a chimeric liver-specific promoter (LSP), an E2F promoter, a telomerase (hTERT) promoter; a cytomegalovirus enhancer/chicken beta-actin/Rabbit β-globin promoter (CAG) promoter, an elongation factor 1-alpha promoter (EF1-alpha) promoter, a human β-glucuronidase promoter, a chicken β-actin (CBA) promoter, a retroviral Rous sarcoma virus (RSV) LTR promoter, a dihydrofolate reductase promoter, and a 13-actin promoter. In some embodiments, the expression construct further comprises an intron. In some embodiments, the intron is a CBA intron or an hEF 1alpha intron. In some embodiments, the intron is a chimeric intron. In some embodiments, the expression vector is a self-complementary vector and the intron is a delta chimeric intron. In some embodiments, the expression construct further comprises a polyadenylation signal. In some embodiments, the polyadenylation signal is a bovine growth hormone polyadenylation signal, an SV40 polyadenylation signal, or a HSV TK polyadenylation signal.

In some embodiments, the disclosure provides a vector comprising any of the expression constructs described herein. In some embodiments, the vector is a recombinant adeno-associated virus (rAAV) vector. In some embodiments, the expression construct is flanked by one or more AAV inverted terminal repeat (ITR) sequences. In some embodiments, the expression construct is flanked by two AAV ITRs. In some embodiments, the AAV ITRs are AAV1, AAV2, AAV3, AAV4, AAV5, AAV6, AAV7, AAV8, AAVrh8, AAVrh8R, AAV9, AAV10, AAVrh10, AAV11, AAV12, AAV2R471A, AAV DJ, a goat AAV, bovine AAV, or mouse AAV serotype ITRs. In some embodiments, the AAV ITRs are AAV2 ITRs. In some embodiments, the vector further comprises a stuffer nucleic acid. In some embodiments, the stuffer nucleic acid is located upstream or downstream of the nucleic acid encoding the artificial miRNA. In some embodiments, the vector is a self-complementary rAAV vector. In some embodiments, the vector comprises first nucleic acid sequence encoding the artificial miRNA and a second nucleic acid sequence encoding a complement of the artificial miRNA, wherein the first nucleic acid sequence can form intrastrand base pairs with the second nucleic acid sequence along most or all of its length. In some embodiments, the first nucleic acid sequence and the second nucleic acid sequence are linked by a mutated AAV ITR, wherein the mutated AAV ITR comprises a deletion of the D region and comprises a mutation of the terminal resolution sequence.

In some embodiments, the disclosure provides a cell comprising any of the rAAV vectors as described herein.

In some embodiments, the disclosure provides a recombinant AAV particle comprising any of the rAAV vectors as described herein. In some embodiments, the AAV viral particle comprises an AAV1, AAV2, AAV3, AAV4, AAV5, AAV6, AAV7, AAV8, AAVrh8, AAVrh8R, AAV9, AAV10, AAVrh10, AAV11, AAV12, AAV2R471A, AAV2/2-7m8, AAV DJ, AAV2 N587A, AAV2 E548A, AAV2 N708A, AAV V708K, AAV2-HBKO, AAVDJ8, AAVPHP.B, AAVPHP.eB, AAVBR1, AAVHSC15, AAVHSC17, a goat AAV, AAV1/AAV2 chimeric, bovine AAV, or mouse AAV capsid rAAV2/HBoV1 serotype capsid. In some embodiments, the ITR and the capsid of the rAAV viral particle are derived from the same AAV serotype. In some embodiments, the ITR and the capsid of the rAAV viral particle are derived from different AAV serotypes. In some embodiments, the ITR is derived from AAV2 and the capsid of the rAAV particle is derived from AAV1.

In some embodiments, the capsid proteins of the rAAV particles are modified AAV9 capsid. In some such embodiments, the modified AAV9 capsid proteins of the AAV viral particles comprise targeting peptides inserted into the AAV9 capsid that alter the transduction and/or endosomal release of the viral particle following administration to the patient. The rAAV particles comprising modified AAV9 capsid proteins, as disclosed herein, comprise three structural capsid proteins, VP1, VP2 and VP3. The three capsid proteins are alternative splice variants. In some embodiments, the targeting peptide is inserted into the VP1, VP2 and VP3 capsid proteins within the rAAV particle.

In particular embodiments, the targeting peptide of the modified AAV9 capsids are inserted after residue 588 of the AAV9 structural protein (numbering based on VP1 numbering of AAV9). In some embodiments, the targeting peptide has SEQ ID NO: 57. In some embodiments, the targeting peptide is flanked by linker sequences on the N-terminal and the C-terminal end of the targeting peptide. In some embodiments, the linker sequence on the N-terminal side has the sequence AAA. In some embodiments, the linker sequence on the C-terminal side is AS. In some embodiments, the full sequence inserted after residue 588 of the AAV9 capsid structural protein has SEQ ID NO: 58. In some embodiments, the full modified AAV9 capsid structural protein has SEQ ID NO: 59. In some embodiments, the full modified AAV9 capsid structural protein that it at least 90% (e.g., at least 92%, at least 95%, at least 98%, at least 98.5%, at least 99%, at least 99.2%, at least 99.5%, or at least 99.8%) identical to SEQ ID NO: 59, wherein the modified AAV9 structural capsid comprises the targeting peptide of SEQ ID NO: 57. The capsid having SEQ ID NO: 59 will also be referred to herein as SAN006 or AAV.SAN006.

In some embodiments, the disclosure provides a composition comprising any of the rAAV particles described herein. In some embodiments, the composition further comprises a pharmaceutically acceptable carrier.

In some embodiments, the disclosure provides a kit comprising any of the artificial miRNA molecules described herein. In some embodiments, the disclosure provides a kit comprising any of the AAV particles described herein. In some embodiments, the disclosure provides a kit comprising any of the compositions described herein. In some embodiments, the kit further comprises instructions for use.

In some aspects, the disclosure provides methods for treating a tauopathy in a patient in need thereof, comprising administering to the patient a composition comprising an artificial miRNA comprising a guide strand that binds to a tau mRNA and a non-guide (passenger) strand, wherein the guide strand and the non-guide have sequences as disclosed herein. In some embodiments, the tauopathy is progressive supranuclear palsy (PSP). In other embodiments, the tauopathy is Alzheimer's disease (AD). In some embodiments, the tauopathy is corticobasal degeneration.

In some aspects, the disclosure provides methods for of reducing tau expression in a patient suffering from a tauopathy, comprising administering to the patient a composition comprising a miRNA comprising a guide strand that binds to a tau mRNA and a non-guide (passenger) strand, wherein the guide strand and the non-guide have sequences as disclosed herein. In some embodiments, administering the artificial miRNA molecules disclosed herein prevents tau aggregation. In some embodiments, administering the artificial miRNA molecules disclosed herein results in reduction of existing tau aggregates.

### BRIEF DESCRIPTION OF THE DRAWINGS

FIG. 1 shows that artificial miRNA sequences disclosed herein reduce tau expression in human cells. U2OS cells stably expressing full-length 4R tau with the G272V and P301S mutations were transfected with the indicated plasmids in triplicate. Cells were lysed after 72 hours of expression and human tau protein was measured by ELISA and normalized to total protein content in each sample. Values represent the mean normalized to control (CTL3)+/− SEM. ** p<0.01, *p<0.05, 1-way ANOVA. The x-axis shows the various artificial miRNA molecules of the disclosure (see Table 1). The y axis shows reduction in tau expression levels.

FIGS. 2A-2B show that artificial miRNA sequences reduce tau expression in a mouse model of tauopathy following intrastriatal AAVrh. 10 injection. Total mRNA and protein from striatal tissue was isolated 4 weeks after delivery of AAVrh. 10-artificial miRNA. MAPT mRNA expression was quantified by TaqMan qPCR (FIG. 2A) and HT7 anti-human tau ELISA was used to quantify human tau protein reduction relative to total protein content (FIG. 2B). Data are mean+/− SEM. * p<0.05, ** p<0.01, *** p<0.001, one way ANOVA.

FIG. 3 shows that artificial miRNA sequences reduce human tau protein expression in a mouse model of tauopathy following intrahippocampal AAVrh.10 injection. Sagittal sections of Tau22 mouse brain were collected 4 weeks post-hippocampal injection of the indicated AAVrh. 10-artificial miRNAs. Tissue was immunostained for human tau and the neuronal marker NeuN, revealing tau reduction in neurons of the dentate gyrus of the hippocampus.

FIG. 4 shows artificial miRNA sequences reduce human tau expression in a mouse model of tauopathy following intrastriatal AAV.SAN006 injection. Total mRNA from striatal tissue was isolated 4 weeks after delivery of AAV.SAN006-artificial miRNA. MAPT mRNA expression was quantified by TaqMan RT-dPCR. Data are mean+/− SEM. ** p<0.01, one way ANOVA.

FIGS. 5A-5B show that artificial miRNA sequences exhibit excellent strand biasing and accurate 5′ processing in vivo. Total striatal RNA was isolated from Tau22 animals treated with AAVrh. 10 vectors expressing the indicated artificial miRNAs and submitted for small RNA sequencing. Three animals from each treatment were analyzed for guide and passenger strand expression (FIG. 5A) and cleavage efficiency of the pre-miRNA (FIG. 5B) to create the predicted mature guide strand. Data are mean+/−SD with the individual animals plotted.

FIGS. 6A-6B show artificial miRNA sequences disclosed herein reduce human tau mRNA and protein in a mouse model of tauopathy following intravenous AAV-PHP.eB injection. Total mRNA and protein were isolated from hindbrain tissue 4 weeks after delivery of AAV-PHP.eB vectors expressing the indicated artificial miRNAs. ddPCR with human MAPT TaqMan assay was used to analyze gene expression (FIG. 6A) and anti-human tau ELISA was used to quantify human tau protein expression (FIG. 6B) from the same samples. Data are mean +/− SEM. ** p<0.01, **** p<0.0001, one-way ANOVA.

FIGS. 7A-7B show artificial miRNA sequences reduce total tau and phospho tau protein in a mouse model of tauopathy using a late-intervention efficacy paradigm. Tissue homogenates were prepared from hindbrain and spinal cord tissue three months after delivery of AAV-PHP.eB vectors expressing the indicated artificial miRNAs in six-month old Tau22 mice. A Quanterix Simoa assay was used to analyze total tau protein (FIG. 7A) and phospho tau 181 protein (FIG. 7B). Data are mean+/− SEM. * p<0.05, ** p<0.01, *** p<0.001 **** p<0.0001, one-way ANOVA.

FIGS. 8A-8D show artificial miRNA sequences reduce phospho tau aggregates in a mouse model of tauopathy using a late-intervention efficacy paradigm. Fixed tissue sections were prepared from cortex and hindbrain three months after delivery of AAV-PHP.eB vectors expressing the indicated artificial miRNAs in six-month old Tau22 mice. Immunohistochemistry was performed on tissues using the AT8 antibody recognizing phospho tau 202/205 (FIG. 8A) and images (FIG. 8B) were analyzed using pixel-based thresholds for weak, moderate, and strong AT8+ signal in cortex (FIG. 8C) and hindbrain (FIG. 8D). Data are mean+/−SD. *p<0.05, ** p<0.01, two-way ANOVA.

FIGS. 9A-9B show artificial miRNA sequences reduce plasma and CSF neurofilament light chain in a mouse model of tauopathy using a late-intervention efficacy paradigm. Plasma and CSF were collected three months after delivery of AAV-PHP.eB vectors expressing the indicated artificial miRNAs in six-month old Tau22 mice. Quanterix Simoa assay was used to measure neurofilament light chain (NfL) in plasma (FIG. 9A) and CSF (FIG. 9B). Data are mean+/− SEM. * p<0.05, ** p<0.01, two-way ANOVA as depicted in FIG. 9A and a: p<0.01, one-way ANOVA as depicted in FIG. 9B.

### DETAILED DESCRIPTION

The microtubule-associated protein tau (MAPT) plays a pathogenic role in a spectrum of devastating and incurable neurodegenerative diseases known as tauopathies. Of these, progressive supranuclear palsy (PSP) is a largely sporadic disease characterized by inclusions of four-repeat tau isoforms in neurons and glial cells and degeneration of subcortical structures. This pathology manifests in severe and progressive motor and cognitive deficits for which there is no approved disease-modifying therapy.

In some aspects, methods for reducing MAPT expression in multiple models of neurological disease may provide safe treatment; may reverse neuropathology, may slow tau aggregate formation and spread; and/or may improve cognitive deficits. RNA interference (RNAi) has shown increasing promise as a therapeutic modality to reduce levels of target mRNA and protein. In some embodiments, the method may comprise using RNAi. In some embodiments, Adeno-associated viruses (AAVs) may be gene delivery vectors configured to express therapeutic constructs for years in the brain. Provided are various embodiments comprising the use of an AAV-RNAi vector for total MAPT lowering for the treatment of tauopathies, such as PSP.

Current treatments for tauopathy are largely symptomatic and supportive, and none have been shown to significantly reduce tau pathology. While many tau-targeted therapeutics are in development, a majority target the tau protein itself. However, targeting the tau protein itself can be problematic, given the heterogeneity of tau isoform expression, posttranslational modification, and conformation between tauopathies.

In some aspects, the disclosure provides methods for targeting the upstream MAPT mRNA with artificial miRNA. Such methods may stand to have a broad therapeutic impact.

In some aspects, the disclosure provides artificial miRNA molecules for treating tauopathy. In some embodiments, the tauopathy is supranuclear palsy (PSP). In some embodiments, the tauopathy is Alzheimer's disease (AD). In some embodiments, the tauopathy is corticobasal degeneration. In some embodiments, the artificial miRNA reduces expression of tau protein.

In some embodiments, the therapeutic constructs described herein may be related to fields comprising RNA inhibition, molecular biology, and/or central nervous system (CNS) gene therapy. In some embodiments, the therapeutic constructs described herein may be designed to reduce expression of the microtubule associated protein tau and may provide a method to treat neurodegenerative tauopathies comprising progressive supranuclear palsy (PSP) and/or Alzheimer's disease (AD).

In some embodiments, the artificial miRNA comprises a guide strand that has a nucleotide sequence having at least about 90% identity or at least about 95% identity to the sequence of SEQ ID NO: 1 (5′-UUCGCGGAAGGUCAGCUUGUG-3′) SEQ ID NO: 51 (5′-XUCGCGGAAGGUCAGCUUGUG-3′), SEQ ID NO: 2 (5′-GACGGCGACUUGGGUGGAGUA-3′), SEQ ID NO: 3 (5′-UGUCGAUGCUGCCGGUGGAGG-3′), SEQ ID NO: 4 (5′-UUUCGACUGGACUCUGUCCUU-3′), SEQ ID NO: 52 (5′-XUUCGACUGGACUCUGUCCUU-3′), SEQ ID NO: 5 (5′-AGUACGGACCACUGCCACCUU-3′), SEQ ID NO: 6 (5′-AGCCGAUCUUGGACUUGACAU-3′), SEQ ID NO: 7 (5′-GUACGUCCCAGCGUGAUCUUC-3′), SEQ ID NO: 8 (5′-AUGUCGAUGCUGCCGGUGGAG-3′), SEQ ID NO: 9 (5′-UUCGACUGGACUCUGUCCUUG-3′), SEQ ID NO: 10 (5′-GGCGACUUGGGUGGAGUACGG-3′), SEQ ID NO: 11 (5′-GGCGACUUGUACACGAUCUCC-3′), SEQ ID NO: 12 (5′-UAUGUCGAUGCUGCCGGUGGA-3′), SEQ ID NO: 53 (5′-XAUGUCGAUGCUGCCGGUGGA-3′), SEQ ID NO: 13 (5′-UAUGCGAGCUUGGGUCACGUG-3′), SEQ ID NO: 54 (5′-XAUGCGAGCUUGGGUCACGUG-3′), SEQ ID NO: 14 (5′-UGUACGUCCCAGCGUGAUCUU-3′), SEQ ID NO: 55 (5′-XGUACGUCCCAGCGUGAUCUU-3′), SEQ ID NO: 15 (5′-GUCGAUGCUGCCGGUGGAGGA-3′), SEQ ID NO: 16 (5′-AACCCGUACGUCCCAGCGUGA-3′), SEQ ID NO: 17 (5′-GUACGGACCACUGCCACCUUC-3′), SEQ ID NO: 18 (5′-GUAGCCGCUGCGAUCCCCUGA-3′), SEQ ID NO: 19 (5′-UGGCGAUCUUCGUUUUACCAU-3′), SEQ ID NO: 20 (5′-UUCGUCAGCUAGCGUGGCGAG-3′), SEQ ID NO: 56 (5′-XUCGUCAGCUAGCGUGGCGAG-3′), SEQ ID NO: 21 (5′-UCUUUGCUUUUACUGACCAUG-3′), or SEQ ID NO: 22 (5′-UCAAGCUUCUCAGAUUUUAC-3′), wherein X is a nucleotide selected from C, A and G.

In some embodiments, the artificial miRNA comprises a second strand (passenger strand) comprising a non-guide region that comprises a nucleotide sequence that is partially complementary to the nucleotide sequence of the guide region. In one embodiment, the guide sequence comprises the sequence of SEQ ID NO: 1 and the non-guide region comprises the sequence of SEQ ID NO: 23. In another embodiment, the guide sequence comprises the sequence of SEQ ID NO: 2 and the non-guide region comprises the sequence of SEQ ID NO: 24. In another embodiment, the guide sequence comprises the sequence of SEQ ID NO: 3 and the non-guide region comprises the sequence of SEQ ID NO: 25. In another embodiment, the guide sequence comprises the sequence of SEQ ID NO: 4 and the non-guide region comprises the sequence of SEQ ID NO: 26. In another embodiment, the guide sequence comprises the sequence of SEQ ID NO: 5 and the non-guide region comprises the sequence of SEQ ID NO: 27. In another embodiment, the guide sequence comprises the sequence of SEQ ID NO: 6 and the non-guide region comprises the sequence of SEQ ID NO: 28. In another embodiment, the guide sequence comprises the sequence of SEQ ID NO: 7 and the non-guide region comprises the sequence of SEQ ID NO: 29. In another embodiment, the guide sequence comprises the sequence of SEQ ID NO: 8 and the non-guide region comprises the sequence of SEQ ID NO: 30. In another embodiment, the guide sequence comprises the sequence of SEQ ID NO: 9 and the non-guide region comprises the sequence of SEQ ID NO: 31. In another embodiment, the guide sequence comprises the sequence of SEQ ID NO: 10 and the non-guide region comprises the sequence of SEQ ID NO: 32. In another embodiment, the guide sequence comprises the sequence of SEQ ID NO: 11 and the non-guide region comprises the sequence of SEQ ID NO: 33. In another embodiment, the guide sequence comprises the sequence of SEQ ID NO: 12 and the non-guide region comprises the sequence of SEQ ID NO: 34. In another embodiment, the guide sequence comprises the sequence of SEQ ID NO: 13 and the non-guide region comprises the sequence of SEQ ID NO: 35. In another embodiment, the guide sequence comprises the sequence of SEQ ID NO: 14 and the non-guide region comprises the sequence of SEQ ID NO: 36. In another embodiment, the guide sequence comprises the sequence of SEQ ID NO: 15 and the non-guide region comprises the sequence of SEQ ID NO: 37. In another embodiment, the guide sequence comprises the sequence of SEQ ID NO: 16 and the non-guide region comprises the sequence of SEQ ID NO: 38. In another embodiment, the guide sequence comprises the sequence of SEQ ID NO: 17 and the non-guide region comprises the sequence of SEQ ID NO: 39. In another embodiment, the guide sequence comprises the sequence of SEQ ID NO: 18 and the non-guide region comprises the sequence of SEQ ID NO: 40. In another embodiment, the guide sequence comprises the sequence of SEQ ID NO: 19 and the non-guide region comprises the sequence of SEQ ID NO: 41. In another embodiment, the guide sequence comprises the sequence of SEQ ID NO: 20 and the non-guide region comprises the sequence of SEQ ID NO: 42. In another embodiment, the guide sequence comprises the sequence of SEQ ID NO: 21 and the non-guide region comprises the sequence of SEQ ID NO: 43. In another embodiment, the guide sequence comprises the sequence of SEQ ID NO: 22 and the non-guide region comprises the sequence of SEQ ID NO: 44.

In one embodiment, the guide sequence comprises the sequence having at least about 90% identity or at least about 95% identity to the sequence of SEQ ID NO: 51 (5′-XUCGCGGAAGGUCAGCUUGUG-3′) and the non-guide region comprises the sequence having at least about 90% identity or at least about 95% identity to the sequence of SEQ ID NO: 23 (5′-CACAAGCUCCUUCCGCGAG-3′). In one such embodiment, the guide sequence comprises the sequence of SEQ ID NO: 51 (5′-XUCGCGGAAGGUCAGCUUGUG-3′) and the non-guide region comprises the sequence of SEQ ID NO: 23 (5′-CACAAGCUCCUUCCGCGAG-3′), wherein X is a nucleotide selected from C, A and G.

In another embodiment, the guide sequence comprises the sequence having at least about 90% identity or at least about 95% identity to the sequence of SEQ ID NO: 52 (5′-XUUCGACUGGACUCUGUCCUU-3′) and the non-guide region comprises the sequence having at least about 90% identity or at least about 95% identity to the sequence of SEQ ID NO: 26 (5′-AAGGACAGUCCAGUCGAAG-3′). In one such embodiment, the guide sequence comprises the sequence of SEQ ID NO: 52 (5′-XUUCGACUGGACUCUGUCCUU-3′) and the non-guide region comprises the sequence of SEQ ID NO: 26 (5′-AAGGACAGUCCAGUCGAAG-3′), wherein X is a nucleotide selected from C, A and G.

In another embodiment, the guide sequence comprises the sequence having at least about 90% identity or at least about 95% identity to the sequence of SEQ ID NO: 53 (5′-XAUGUCGAUGCUGCCGGUGGA-3′) and the non-guide region comprises the sequence having at least about 90% identity or at least about 95% identity to the sequence of SEQ ID NO: 34 (5′-UCCACCGGGCAUCGACAUG-3′). In one such embodiment, the guide sequence comprises the sequence of SEQ ID NO: 53 (5′-XAUGUCGAUGCUGCCGGUGGA-3′) and the non-guide region comprises the sequence of SEQ ID NO: 34 (5′-UCCACCGGGCAUCGACAUG-3′), wherein X is a nucleotide selected from C, A and G.

In another embodiment, the guide sequence comprises the sequence having at least about 90% identity or at least about 95% identity to the sequence of SEQ ID NO: 54 (5′-XAUGCGAGCUUGGGUCACGUG-3′) and the non-guide region comprises the sequence having at least about 90% identity or at least about 95% identity to the sequence of SEQ ID NO: 35 (5′-CACGUGACAAGCUCGCAUG-3′). In one such embodiment, the guide sequence comprises the sequence of SEQ ID NO: 54 (5′-XAUGCGAGCUUGGGUCACGUG-3′) and the non-guide region comprises the sequence of SEQ ID NO: 35 (5′-CACGUGACAAGCUCGCAUG-3′), wherein X is a nucleotide selected from C, A and G.

In another embodiment, the guide sequence comprises the sequence having at least about 90% identity or at least about 95% identity to the sequence of SEQ ID NO: 55 (5′-XGUACGUCCCAGCGUGAUCUU-3′) and the non-guide region comprises the sequence having at least about 90% identity or at least about 95% identity to the sequence of SEQ ID NO: 36 (5′-AAGAUCACUGGGACGUAUG-3′). In one such embodiment, the guide sequence comprises the sequence of SEQ ID NO: 55 (5′-XGUACGUCCCAGCGUGAUCUU-3′) and the non-guide region comprises the sequence of SEQ ID NO: 36 (5′-AAGAUCACUGGGACGUAUG-3′), wherein X is a nucleotide selected from C, A and G.

In another embodiment, the guide sequence comprises the sequence having at least about 90% identity or at least about 95% identity to the sequence of SEQ ID NO: 56 (5′-XUCGUCAGCUAGCGUGGCGAG-3′) and the non-guide region comprises the sequence having at least about 90% identity or at least about 95% identity to the sequence of SEQ ID NO: 42 (5′-CUCGCCACUAGCUGACGAG-3′). In one such embodiment, the guide sequence comprises the sequence of SEQ ID NO: 56 (5′-XUCGUCAGCUAGCGUGGCGAG-3′) and the non-guide region comprises the sequence of SEQ ID NO: 42 (5′-CUCGCCACUAGCUGACGAG-3′), wherein X is a nucleotide selected from C, A and G.

In some aspects, the disclosure provides expression constructs, vectors (e.g., recombinant AAV vectors), cells, viral particles (e.g., AAV particles), and pharmaceutical compositions comprising an artificial miRNA of the present disclosure. In further aspects, the disclosure provides methods for a tauopathy in a mammal comprising administering to the mammal a pharmaceutical composition comprising an artificial miRNA of the present disclosure. In some embodiments, the tauopathy is supranuclear palsy (PSP), Alzheimer's disease (AD) or corticobasal degeneration.

### I. General Techniques

The techniques and procedures described or referenced herein are generally well understood and commonly employed using conventional methodology by those skilled in the art, such as, for example, the widely utilized methodologies described in Molecular Cloning: A Laboratory Manual (Sambrook et al., 4^{th} ed., Cold Spring Harbor Laboratory Press, Cold Spring Harbor, N.Y., 2012); Current Protocols in Molecular Biology (F. M. Ausubel, et al. eds., 2003); the series Methods in Enzymology (Academic Press, Inc.); POR 2: A Practical Approach (M. J. MacPherson, B. D. Hames and G. R. Taylor eds., 1995); Antibodies, A Laboratory Manual (Harlow and Lane, eds., 1988); Culture of Animal Cells: A Manual of Basic Technique and Specialized Applications (R.I. Freshney, 6^{th} ed., J. Wiley and Sons, 2010); Oligonucleotide Synthesis (M. J. Gait, ed., 1984); Methods in Molecular Biology, Humana Press; Cell Biology: A Laboratory Notebook (J. E. Cellis, ed., Academic Press, 1998); Introduction to Cell and Tissue Culture (J. P. Mather and P. E. Roberts, Plenum Press, 1998); Cell and Tissue Culture: Laboratory Procedures (A. Doyle, J. B. Griffiths, and D. G. Newell, eds., J. Wiley and Sons, 1993-8); Handbook of Experimental Immunology (D. M. Weir and C. C. Blackwell, eds., 1996); Gene Transfer Vectors for Mammalian Cells (J. M. Miller and M. P. Calos, eds., 1987); POR: The Polymerase Chain Reaction, (Mullis et al., eds., 1994); Current Protocols in Immunology (J. E. Coligan et al., eds., 1991); Short Protocols in Molecular Biology (Ausubel et al., eds., J. Wiley and Sons, 2002); Immunobiology (C.A. Janeway et al., 2004); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane, Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 2011).

### II. Definitions

A “vector,” as used herein, refers to a recombinant plasmid or virus that comprises a nucleic acid to be delivered into a host cell, either in vitro or in vivo.

The term “polynucleotide” or “nucleic acid” as used herein refers to a polymeric form of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and thus can be an oligodeoxynucleoside phosphoramidate (P—NH₂) or a mixed phosphoramidate-phosphodiester oligomer. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

A “recombinant viral vector” refers to a recombinant polynucleotide vector comprising one or more heterologous sequences (i.e., nucleic acid sequence not of viral origin). In the case of recombinant AAV vectors, the recombinant nucleic acid is flanked by at least one and in some embodiments two, inverted terminal repeat sequences (ITRs).

A “recombinant AAV vector (rAAV vector)” refers to a polynucleotide vector comprising one or more heterologous sequences (i.e., nucleic acid sequence not of AAV origin) that are flanked by at least one, and in embodiments two, AAV inverted terminal repeat sequences (ITRs). Such rAAV vectors can be replicated and packaged into infectious viral particles when present in a host cell that has been infected with a suitable helper virus (or that is expressing suitable helper functions) and that is expressing AAV rep and cap gene products (i.e. AAV Rep and Cap proteins). When a rAAV vector is incorporated into a larger polynucleotide (e.g., in a chromosome or in another vector such as a plasmid used for cloning or transfection), then the rAAV vector may be referred to as a “pro-vector” which can be “rescued” by replication and encapsidation in the presence of AAV packaging functions and suitable helper functions. An rAAV vector can be in any of a number of forms, including, but not limited to, plasmids, linear artificial chromosomes, complexed with lipids, encapsulated within liposomes, and encapsidated in a viral particle, particularly an AAV particle. A rAAV vector can be packaged into an AAV virus capsid to generate a “recombinant adeno-associated viral particle (rAAV particle)”.

“Heterologous” means derived from a genotypically distinct entity from that of the rest of the entity to which it is compared or into which it is introduced or incorporated. For example, a polynucleotide introduced by genetic engineering techniques into a different cell type is a heterologous polynucleotide (and, when expressed, can encode a heterologous polypeptide). Similarly, a cellular sequence (e.g., a gene or portion thereof) that is incorporated into a viral vector is a heterologous nucleotide sequence with respect to the vector.

The term “transgene” refers to a polynucleotide that is introduced into a cell and is capable of being transcribed into RNA and optionally, translated and/or expressed under appropriate conditions. In aspects, it confers a desired property to a cell into which it was introduced, or otherwise leads to a desired therapeutic or diagnostic outcome. In another aspect, it may be transcribed into a molecule that mediates RNA interference, such as miRNA, siRNA, or shRNA.

“Chicken β-actin (CBA) promoter” refers to a polynucleotide sequence derived from a chicken β-actin gene (e.g., Gallus gallus beta actin, represented by GenBank Entrez Gene ID 396526). As used herein, “chicken β-actin promoter” may refer to a promoter containing a cytomegalovirus (CMV) early enhancer element, the promoter and first exon and intron of the chicken β-actin gene, and the splice acceptor of the rabbit beta-globin gene, such as the sequences described in Miyazaki, J. et al. (1989) Gene 79 (2): 269-77. As used herein, the term “CAG promoter” may be used interchangeably. As used herein, the term “CMV early enhancer/chicken beta actin (CAG) promoter” may be used interchangeably.

The terms “genome particles (gp),” “genome equivalents,” or “genome copies” as used in reference to a viral titer, refer to the number of virions containing the recombinant AAV DNA genome, regardless of infectivity or functionality. The number of genome particles in a particular vector preparation can be measured by procedures such as described in the Examples herein, or for example, in Clark et al. (1999) Hum. Gene Ther., 10:1031-1039; Veldwijk et al. (2002) Mol. Ther., 6:272-278.

The term “vector genome (vg)” as used herein may refer to one or more polynucleotides comprising a set of the polynucleotide sequences of a vector, e.g., a viral vector. A vector genome may be encapsidated in a viral particle. Depending on the particular viral vector, a vector genome may comprise single-stranded DNA, double-stranded DNA, or single-stranded RNA, or double-stranded RNA. A vector genome may include endogenous sequences associated with a particular viral vector and/or any heterologous sequences inserted into a particular viral vector through recombinant techniques. For example, a recombinant AAV vector genome may include at least one ITR sequence flanking a promoter, a stuffer, a sequence of interest (e.g., a miRNA), and a polyadenylation sequence. A complete vector genome may include a complete set of the polynucleotide sequences of a vector. In some embodiments, the nucleic acid titer of a viral vector may be measured in terms of vg/mL. Methods suitable for measuring this titer are known in the art (e.g., quantitative PCR).

As used herein, the term “inhibit” may refer to the act of blocking, reducing, eliminating, or otherwise antagonizing the presence, or an activity of, a particular target. Inhibition may refer to partial inhibition or complete inhibition. For example, inhibiting the expression of a gene may refer to any act leading to a blockade, reduction, elimination, or any other antagonism of expression of the gene, including reduction of mRNA abundance (e.g., silencing mRNA transcription), degradation of mRNA, inhibition of mRNA translation, and so forth. In some embodiments, inhibiting the expression of Tau protein may refer a blockade, reduction, elimination, or any other antagonism of expression of Tau protein, including reduction of Tau mRNA abundance (e.g., silencing Tau mRNA transcription), degradation of Tau mRNA, inhibition of Tau mRNA translation, and so forth. As another example, inhibiting the accumulation of a protein in a cell may refer to any act leading to a blockade, reduction, elimination, or other antagonism of expression of the protein, including reduction of mRNA abundance (e.g., silencing mRNA transcription), degradation of mRNA, inhibition of mRNA translation, degradation of the protein, and so forth. In some embodiments, inhibiting the accumulation of Tau protein in a cell refers to a blockade, reduction, elimination, or other antagonism of expression of the Tau protein in a cell, including reduction of Tau mRNA abundance (e.g., silencing Tau mRNA transcription), degradation of Tau mRNA, inhibition of Tau mRNA translation, degradation of the Tau protein, and so forth

The terms “infection unit (iu),” “infectious particle,” or “replication unit,” as used in reference to a viral titer, refer to the number of infectious and replication-competent recombinant AAV vector particles as measured by the infectious center assay, also known as replication center assay, as described, for example, in Mclaughlin et al. (1988) J. Virol., 62:1963-1973.

The term “transducing unit (tu)” as used in reference to a viral titer, refers to the number of infectious recombinant AAV vector particles that result in the production of a functional transgene product as measured in functional assays such as described in Examples herein, or for example, in Xiao et al. (1997) Exp. Neurobiol., 144:113-124; or in Fisher et al. (1996) J. Virol., 70:520-532 (LFU assay).

An “inverted terminal repeat” or “ITR” sequence is a term well understood in the art and refers to relatively short sequences found at the termini of viral genomes which are in opposite orientation.

An “AAV inverted terminal repeat (ITR)” sequence, a term well-understood in the art, is an approximately 145-nucleotide sequence that is present at both termini of the native single-stranded AAV genome. The outermost 125 nucleotides of the ITR can be present in either of two alternative orientations, leading to heterogeneity between different AAV genomes and between the two ends of a single AAV genome. The outermost 125 nucleotides also contains several shorter regions of self-complementarity (designated A, A′, B, B′, C, C′ and D regions), allowing intrastrand base-pairing to occur within this portion of the ITR.

A “terminal resolution sequence” or “trs” is a sequence in the D region of the AAV ITR that is cleaved by AAV rep proteins during viral DNA replication. A mutant terminal resolution sequence is refractory to cleavage by AAV rep proteins.

“AAV helper functions” refer to functions that allow AAV to be replicated and packaged by a host cell. AAV helper functions can be provided in any of a number of forms, including, but not limited to, helper virus or helper virus genes which aid in AAV replication and packaging. Other AAV helper functions are known in the art such as genotoxic agents.

A “helper virus” for AAV refers to a virus that allows AAV (which is a defective parvovirus) to be replicated and packaged by a host cell. A helper virus provides “helper functions” which allow for the replication of AAV. A number of such helper viruses have been identified, including adenoviruses, herpesviruses and, poxviruses such as vaccinia and baculovirus. The adenoviruses encompass a number of different subgroups, although Adenovirus type 5 of subgroup C (Ad5) is most commonly used. Numerous adenoviruses of human, non-human mammalian and avian origin are known and are available from depositories such as the ATCC. Viruses of the herpes family, which are also available from depositories such as ATCC, include, for example, herpes simplex viruses (HSV), Epstein-Barr viruses (EBV), cytomegaloviruses (CMV) and pseudorabies viruses (PRV). Examples of adenovirus helper functions for the replication of AAV include ElA functions, ElB functions, E2A functions, VA functions and E4orf6 functions. Baculoviruses available from depositories include Autographa californica nuclear polyhedrosis virus.

A preparation of rAAV is said to be “substantially free” of helper virus if the ratio of infectious AAV particles to infectious helper virus particles is at least about 10²:1; at least about 10⁴:1, at least about 10⁶:1; or at least about 10⁸:1 or more. In some embodiments, preparations are also free of equivalent amounts of helper virus proteins (i.e., proteins as would be present as a result of such a level of helper virus if the helper virus particle impurities noted above were present in disrupted form). Viral and/or cellular protein contamination can generally be observed as the presence of Coomassie staining bands on SDS gels (e.g., the appearance of bands other than those corresponding to the AAV capsid proteins VP1, VP2 and VP3).

“Percent (%) sequence identity” with respect to a reference polypeptide or nucleic acid sequence is defined as the percentage of amino acid residues or nucleotides in a candidate sequence that are identical with the amino acid residues or nucleotides in the reference polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various ways that are within the skill in the art, for instance, using publicly available computer software programs, for example, those described in Current Protocols in Molecular Biology (Ausubel et al., eds., 1987), Supp. 30, section 7.7.18, Table 7.7.1, and including BLAST, BLAST-2, ALIGN or Megalign (DNASTAR) software. A preferred alignment program is ALIGN Plus (Scientific and Educational Software, Pennsylvania). Those skilled in the art can determine appropriate parameters for measuring alignment, including any algorithms needed to achieve maximal alignment over the full length of the sequences being compared. For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. For purposes herein, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows: 100 times the fraction W/Z, where W is the number of nucleotides scored as identical matches by the sequence alignment program in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C.

An “isolated” molecule (e.g., nucleic acid or protein) or cell means it has been identified and separated and/or recovered from a component of its natural environment.

An “effective amount” is an amount sufficient to effect beneficial or desired results, including clinical results (e.g., amelioration of symptoms, achievement of clinical endpoints, and the like). An effective amount can be administered in one or more administrations. In terms of a disease state, an effective amount is an amount sufficient to ameliorate, stabilize, or delay development of a disease.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

As used herein, “treatment” is an approach for obtaining beneficial or desired clinical results. For purposes of this disclosure, beneficial or desired clinical results include, but are not limited to, alleviation of symptoms, diminishment of extent of disease, stabilized (e.g., not worsening) state of disease, preventing spread (e.g., metastasis) of disease, delay or slowing of disease progression, amelioration or palliation of the disease state, and remission (whether partial or total), whether detectable or undetectable. “Treatment” can also mean prolonging survival as compared to expected survival if not receiving treatment.

As used herein, the term “prophylactic treatment” refers to treatment, wherein an individual is known or suspected to have or be at risk for having a disorder but has displayed no symptoms or minimal symptoms of the disorder. An individual undergoing prophylactic treatment may be treated prior to onset of symptoms.

As used herein, the term “tauopathy” refers to a heterogeneous neurodegenerative disorder characterized by the accumulation of phosphorylated and misfolded tau protein in the brain parenchyma. Examples include, but are not limited to Alzheimer's disease (AD), progressive supranuclear palsy (PSP), corticobasal degeneration (CBD), frontotemporal dementia with parkinsonism-17 (FTDP-17), Pick's Disease (PiD), argyrophilic grain disease (AGD), globular glial tauopathy (GGT), chronic traumatic encephalopathy and post-encephalitic parkinsonism.

“miRNA scaffold” may refer to a polynucleotide containing (i) a double-stranded sequence targeting a gene of interest for knockdown by miRNA and (ii) additional sequences that form a stem-loop structure resembling that of endogenous miRNAs. A sequence targeting a gene of interest for miRNA (e.g., a short, ˜20-nt sequence) may be ligated to sequences that create a miRNA-like stem-loop and a sequence that base pairs with the sequence of interest to form a duplex when the polynucleotide is assembled into the miRNA-like secondary structure. As described herein, this duplex may hybridize imperfectly, e.g., it may contain one or more unpaired or mispaired bases. Upon cleavage of this polynucleotide by Dicer, this duplex containing the sequence targeting a gene of interest may be unwound and incorporated into the RISC complex. A miRNA scaffold may refer to the miRNA itself or to a DNA polynucleotide encoding the miRNA. An example of a miRNA scaffold is the miR-155 sequence (Lagos-Quintana, M. et al. (2002) Curr. Biol. 12:735-9). Commercially available kits for cloning a sequence into a miRNA scaffold are known in the art (e.g., the Invitrogen™ BLOCK-iT™ Pol II miR RNA interference expression vector kit from Life Technologies, Thermo Fisher Scientific; Waltham, MA).

As used herein, a “bulge” refers to a region of nucleic acid that is non-complementary to nucleic acid opposite it in a duplex nucleic acid. For example, a bulge may refer to a nucleic acid sequence that is noncomplementary to nucleic acid opposite in a duplex nucleic acid where the bulge is flanked by regions of nucleic acid that are complementary to nucleic acid opposite in a duplex nucleic acid. In some examples, the bulge may be any of 1, 2, 3, 4, 5, 6, 7, 8, 9, 10 or greater than 10 bases in length. In some examples, the bulge may be the result of mispairing (e.g., the opposite strand contains a base that is noncomplementary) or the bulge may be the result of nonpairing (e.g., the opposite strand comprises nucleic acid complementary to nucleic acid flanking the bulge but the opposite strand does not contain nucleic acid opposite the bulge).

As used herein, the term “sense” nucleic acid is a nucleic acid comprising a sequence that encodes all or a part of a transgene. In some examples, mRNA for a transgene is a sense nucleic acid.

As used herein, “antisense” nucleic acid is a sequence of nucleic acid that is complementary to a “sense” nucleic acid. For example, an antisense nucleic acid may be complementary to an mRNA encoding a transgene.

As used herein, the “guide region” of a miRNA is the strand of the miRNA that binds the target mRNA, typically on the basis of complementarity. The region of complementarity may encompass the all or a portion of the guide region. Typically, the region of complementarity includes at least the seed region. In many cases, the antisense region of an miRNA is the guide region.

As used herein, the “passenger region,” or “non-guide region,” used interchangeably herein, of an miRNA is the region of the miRNA that is complementary to the guide region. In many cases, the sense region of an miRNA is the passenger region.

As used herein, the “seed region” of an miRNA is a region of about 1-8 nucleotides in length of a miRNA. In some examples, the seed region and the 3′-UTR of its target mRNA may be a key determinant in miRNA recognition.

As used herein, “off-target gene silencing” refers to the pairing of a seed region of a miRNA with sequences in 3′-UTRs of unintended mRNAs and directs translational repression and destabilization of those transcripts (e.g., reduces expression of the unintended mRNAs).

Reference to “about” a value or parameter herein includes (and describes) embodiments that are directed to that value or parameter per se. For example, description referring to “about X” includes description of “X.”

As used herein, the singular form of the articles “a,” “an,” and “the” includes plural references unless indicated otherwise.

It is understood that aspects and embodiments of the disclosure described herein include “comprising,” “consisting,” and/or “consisting essentially of” aspects and embodiments. III. Artificial miRNA Molecules

In some aspects, the disclosure provides artificial miRNA molecules comprising sequences disclosed herein. A miRNA is known in the art as an RNA molecule that induces RNA interference in a cell comprising a short (e.g., 19-25 base pairs) sequence of double-stranded RNA linked by a loop and containing one or more additional sequences of double-stranded RNA comprising one or more bulges (e.g., mispaired or unpaired base pairs). In some embodiments, “miRNA” may refer to a pri-miRNA or a pre-miRNA. During miRNA processing, a pri-miRNA transcript is produced. The pri-miRNA is processed by Drosha-DGCR8 to produce a pre-miRNA by excising one or more sequences to leave a pre-miRNA with a 5′flanking region, a guide strand, a loop region, a non-guide strand, and a 3′flanking region; or a 5′flanking region, a non-guide strand, a loop region, a guide strand, and a 3′flanking region. The pre-miRNA is then exported to the cytoplasm and processed by Dicer to yield a miRNA with a guide strand and a non-guide (or passenger) strand. The guide strand is then used by the RISC complex to catalyze gene silencing, e.g., by recognizing a target RNA sequence complementary to the guide strand. The recognition of a target sequence by a miRNA is primarily determined by pairing between the target and the miRNA seed sequence, e.g., nucleotides 1-8 (5′ to 3′) of the guide strand (see, e.g., Boudreau, R. L. et al. (2013) Nucleic Acids Res. 41: e9).

In the pri/pre-miRNA structure, the guide strand: non-guide strand interface in a duplex is formed in part through complementary base pairing (e.g., Watson-Crick base pairing). However, in some embodiments, this complementary base pairing does not extend through the entire duplex. In some embodiments, a bulge in the interface may exist at one or more nucleotide positions. As used herein, the term “bulge” may refer to a region of nucleic acid that is non-complementary to the nucleic acid opposite it in a duplex. In some embodiments, the bulge is formed when the regions of complementary nucleic acids bind to each other, whereas the regions of central non-complementary region do not bind. In some embodiments, the bulge is formed when the two strands of nucleic acid positioned between the two complementary regions are of different lengths. As described below, a bulge May 1 or more nucleotides. In some embodiments, the miRNA comprises an internal bulge generated by deleting 2 based from the passenger strand of the miRNA-bases 9-10, counting from the start of the passenger strand.

In a particular aspect, the artificial miRNA molecules described in this disclosure are inhibitory against tau mRNA. In some embodiments, the tau mRNA is human tau mRNA. In some embodiments, the artificial mRNA targets the coding sequence of the tau mRNA. In some embodiments, the artificial miRNA targets the 3′-UTR region of mRNA encoding tau. In some embodiments, the artificial miRNA inhibits the expression of tau in a subject. In some embodiments, the artificial miRNA inhibits the accumulation of tau protein in a subject. In some embodiments, the subject is a mammal. In some embodiments, the mammal is a human.

The safety of miRNA-based therapies can be hampered by the ability of the miRNAs to bind to unintended mRNAs and reduce their expression, an effect known as off-target gene silencing. Off-targeting primarily occurs when the seed region (nucleotides 2-8 of the small miRNA) pairs with sequences in 3′-UTRs of unintended mRNAs and directs translational repression and destabilization of those transcripts. Reduced off-targeting miRNA may be designed by substituting bases within the guide and nonguide sequences; e.g., by creating CpG motifs. Potential substitutions that may result in a significantly lower off-target score can be evaluated using the SiSPOTR algorithm, a specificity-focused design algorithm which identifies candidate sequences with minimal off-targeting potentials and potent silencing capacities (Boudreau et al, Nucleic Acids Res. 2013 January; 41 (1) e9. A reduced SiSPOTR score predicts sequences that have a lower number of potential human off targets compared parent miRNA molecules. In some embodiments of the disclosure, the miRNA is improved to reduce off-target gene silencing. In some embodiments, the miRNA comprises one or more CpG motifs. In some embodiments, the miRNA comprises one or more CpG motifs in a seed region.

In some embodiments, the first strand and the second strand are linked by means of a RNA (e.g., a RNA linker) capable of forming a loop structure. As is commonly known in the art, an RNA loop structure (e.g., a stem-loop or hairpin) is formed when an RNA molecule comprises two sequences of RNA that base pair together separated by a sequence of RNA that does not base pair together. For example, a loop structure may form in the RNA molecule A-B-C if sequences A and C are complementary or partially complementary such that they base pair together, but the bases in sequence B do not base pair together. In some embodiments, the loop sequence is 5′-GTTTTGGCCACTGACTGAC-3′ (SEQ ID NO: 45) in DNA form or 5′-GUUUUGGCCACUGACUGAC-3′ (SEQ ID NO: 46) in RNA form.

In some embodiments, the RNA capable of forming a loop structure comprises from 4 to 50 nucleotides. In certain embodiments, the RNA capable of forming a loop structure comprises 13 nucleotides. In some embodiments, the number of nucleotides in the RNA capable of forming a loop is from 4 to 50 nucleotides or any integer there between. In some embodiments, from 0-50% of the loop can be complementary to another portion of the loop. As used herein, the term “loop structure” is a sequence that joins two complementary strands of nucleic acid. In some embodiments, 1-3 nucleotides of the loop structure are contiguous to the complementary strands of nucleic acid and may be complementary to 1-3 nucleotides of the distal portion of the loop structure. For example, the three nucleotides at the 5′ end of the loop structure may be complementary to the three nucleotides at the 3′ end of the loop structure.

In some embodiments, nucleic acid encoding a miRNA of the present disclosure comprises a heterologous miRNA scaffold. In some embodiments, use of a heterologous miRNA scaffold is used to modulate miRNA expression; for example, to increase miRNA expression or to decrease miRNA expression. Any miRNA scaffold known in the art may be used. In some embodiments, the miRNA scaffold is derived from a miR-155 scaffold (see, e.g., Lagos-Quintana, M. et al. (2002) Curr. Biol. 12:735-9 and the Invitrogen™ BLOCK-iT™ Pol II miR RNA interference expression vector kit from Life Technologies, Thermo Fisher Scientific; Waltham, MA).

In some embodiments, the miRNA is selected from Table 1.

In some embodiments, the first strand comprises a nucleic acid sequence having more than about any of 75%, 80%, 85%, 90%, 95%, 96%, 97%, 98% or 99% identity to any guide sequences. In some embodiments, the first strand comprises a nucleic acid sequence having more than about any of 75%, 80%, 85%, 90%, 95%, 96%, 97%, 98% or 99% identity to any guide sequences but maintains the CpG motif. In some embodiments, the second strand comprises a nucleic acid sequence having more than about any of 75%, 80%, 85%, 90%, 95%, 96%, 97%, 98% or 99% identity to the corresponding passenger sequence. In some embodiments, the second strand comprises a nucleic acid sequence having more than about any of 75%, 80%, 85%, 90%, 95%, 96%, 97%, 98% or 99% identity to the corresponding passenger sequence but maintains the CpG motif.

TABLE 1

| miRNA | | |
|---|---|---|
| ID | Guide strand 5′-3′ | Passenger strand 5′-3′ |
| Tau01 | UUCGCGGAAGGUCAGCUUGUG | CACAAGCUCCUUCCGCGAG |
| | (SEQ ID NO: 1) | (SEQ ID NO: 23) |
| Tau 02 | GACGGCGACUUGGGUGGAGUA | UACUCCACAAGUCGCCGUU |
| | (SEQ ID NO: 2) | (SEQ ID NO: 24) |
| Tau 03 | UGUCGAUGCUGCCGGUGGAGG | CCUCCACCCAGCAUCGAUA |
| | (SEQ ID NO: 3) | (SEQ ID NO: 25) |
| Tau 04 | UUUCGACUGGACUCUGUCCUU | AAGGACAGUCCAGUCGAAG |
| | (SEQ ID NO: 4) | (SEQ ID NO: 26) |
| Tau 05 | AGUACGGACCACUGCCACCUU | AAGGUGGCUGGUCCGUAUU |
| | (SEQ ID NO: 5) | (SEQ ID NO: 27) |
| Tau 06 | AGCCGAUCUUGGACUUGACAU | AUGUCAAGCAAGAUCGGUU |
| | (SEQ ID NO: 6) | (SEQ ID NO: 28) |
| Tau 07 | GUACGUCCCAGCGUGAUCUUC | GAAGAUCACUGGGACGUAU |
| | (SEQ ID NO: 7) | (SEQ ID NO: 29) |
| Tau 08 | AUGUCGAUGCUGCCGGUGGAG | CUCCACCGAGCAUCGAUAU |
| | (SEQ ID NO: 8) | (SEQ ID NO: 30) |
| Tau 09 | UUCGACUGGACUCUGUCCUUG | CAAGGACAGUCCAGUCGAA |
| | (SEQ ID NO: 9) | (SEQ ID NO: 31) |
| Tau 10 | GGCGACUUGGGUGGAGUACGG | CCGUACUCCCCAAGUCGUU |
| | (SEQ ID NO: 10) | (SEQ ID NO: 32) |
| Tau 11 | GGCGACUUGUACACGAUCUCC | GGAGAUCGUACAAGUCGUU |
| | (SEQ ID NO: 11) | (SEQ ID NO: 33) |
| Tau 12 | UAUGUCGAUGCUGCCGGUGGA | UCCACCGGGCAUCGACAUG |
| | (SEQ ID NO: 12) | (SEQ ID NO: 34) |
| Tau 13 | UAUGCGAGCUUGGGUCACGUG | CACGUGACAAGCUCGCAUG |
| | (SEQ ID NO: 13) | (SEQ ID NO: 35) |
| Tau 14 | UGUACGUCCCAGCGUGAUCUU | AAGAUCACUGGGACGUAUG |
| | (SEQ ID NO: 14) | (SEQ ID NO: 36) |
| Tau 15 | GUCGAUGCUGCCGGUGGAGGA | UCCUCCACGCAGCAUCGAU |
| | (SEQ ID NO: 15) | (SEQ ID NO: 37) |
| Tau 16 | AACCCGUACGUCCCAGCGUGA | UCACGCUGACGUACGGGUU |
| | (SEQ ID NO: 16) | (SEQ ID NO: 38) |
| Tau 17 | GUACGGACCACUGCCACCUUC | GAAGGUGGGUGGUCCGUAU |
| | (SEQ ID NO: 17) | (SEQ ID NO: 39) |
| Tau 18 | GUAGCCGCUGCGAUCCCCUGA | UCAGGGGAGCAGCGGCUAU |
| | (SEQ ID NO: 18) | (SEQ ID NO: 40) |
| Tau 19 | UGGCGAUCUUCGUUUUACCAU | AUGGUAAAGAAGAUCGUUA |
| | (SEQ ID NO: 19) | (SEQ ID NO: 41) |
| Tau 20 | UUCGUCAGCUAGCGUGGCGAG | CUCGCCACUAGCUGACGAG |
| | (SEQ ID NO: 20) | (SEQ ID NO: 42) |
| Tau 21 | UCUUUGCUUUUACUGACCAUG | CAUGGUCAAAAAGCAAAGA |
| | (SEQ ID NO: 21) | (SEQ ID NO: 43) |
| Tau 22 | UCAAGCUUCUCAGAUUUUAC | GUAAAAUCAGAAGCUUGA |
| | (SEQ ID NO: 22) | (SEQ ID NO: 44) |

IV. miRNA Expression Constructs and Vectors

The disclosure provides expression constructs, vectors and viral particles for expression of the miRNA molecules described herein.

In some embodiments, nucleic acid encoding an artificial miRNA of the present disclosure comprises a heterologous miRNA scaffold. In some embodiments, use of a heterologous miRNA scaffold is used to modulate miRNA expression; for example, to increase miRNA expression or to decrease miRNA expression. Any miRNA scaffold known in the art may be used. In some embodiments, the miRNA scaffold is derived from a miR-155 scaffold (see, e.g., Lagos-Quintana, M. et al. (2002) Curr. Biol. 12:735-9 and the Invitrogen™ BLOCK-iT™ Pol II miR RNA interference expression vector kit from Life Technologies, Thermo Fisher Scientific; Waltham, MA). In some embodiments, nucleic acid encoding a miRNA of the present disclosure comprises a miRNA scaffold. In some embodiments, miRNA scaffold comprises the sequence

(SEQ ID NO: 47)
ctggaggcttgctgaaggctgtatgctgcaggacacaaggcctgttact
agcactcacatggaacaaatggc,

wherein the miRNA is inserted between the bolded gc residues.

In some embodiments, the miRNA in the scaffold comprises the sequence

(SEQ ID NO: 48)
ctggaggcttgctgaaggctgtatgctgtacgatctaatatcgctcgtt
ttggccactgactgacgagcgatatgatcgtacgacaggacacaaggcc
tgttactagcactcacatggaacaaatggc

where the underlined regular text represents the 5′-flank, italics text represents the guide sequence, bolded text represents the loop, underlined italics represents the non-guide sequence and regular text represents the 3′ flank.

In some embodiments, the miRNA targets RNA encoding a polypeptide associated with a tauopathy. In some embodiments, the polypeptide is tau.

In some embodiments, the transgene (e.g., a miRNA of the present disclosure) is operably linked to a promoter. Exemplary promoters include, but are not limited to, the cytomegalovirus (CMV) immediate early promoter, the RSV LTR, the MoML V LTR, the phosphoglycerate kinase-1 (PGK) promoter, a simian virus 40 (SV40) promoter and a CK6 promoter, a transthyretin promoter (TTR), a TK promoter, a tetracycline responsive promoter (TRE), an HBV promoter, an hAAT promoter, a LSP promoter, chimeric liver-specific promoters (LSPs), the E2F promoter, the telomerase (hTERT) promoter; the cytomegalovirus enhancer/chicken beta-actin/Rabbit β-globin promoter (CAG promoter; Niwa et al., Gene, 1991, 10⁸ (2): 193-9) and the elongation factor 1-alpha promoter (EF1-alpha) promoter (Kim et al., Gene, 1990, 91 (2): 217-23 and Guo et al., Gene Ther., 1996, 3 (9): 802-10). In some embodiments, the promoter comprises a human β-glucuronidase promoter or a cytomegalovirus enhancer linked to a chicken β-actin (CBA) promoter. The promoter can be a constitutive, inducible or repressible promoter. In some embodiments, the disclosure provides a recombinant vector comprising nucleic acid encoding a heterologous transgene of the present disclosure operably linked to a CBA promoter. Exemplary promoters and descriptions may be found, e.g., in U.S. PG Pub. 20140335054. In some embodiments, the promoter is a CBA promoter, a minimum CBA promoter, a CMV promoter or a GUSB promoter. In some embodiments, the promoter is a hEF1a promoter.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the 13-actin promoter, the phosphoglycerol kinase (PGK) promoter, and the EF1a promoter [Invitrogen].

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al, Proc. Natl. Acad. Sci. USA, 93:3346-3351 (1996)), the tetracycline-repressible system (Gossen et al, Proc. Natl. Acad. Sci. USA, 89:5547-5551 (1992)), the tetracycline-inducible system (Gossen et al, Science, 268:1766-1769 (1995), see also Harvey et al, Curr. Opin. Chem. Biol., 2:512-518 (1998)), the RU486-inducible system (Wang et al, Nat. Biotech., 15:239-243 (1997) and Wang et al, Gene Ther., 4:432-441 (1997)) and the rapamycin-inducible system (Magari et al, J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

In another embodiment, the native promoter, or fragment thereof, for the transgene will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. Exemplary tissue-specific regulatory sequences include, but are not limited to the following tissue specific promoters: neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)). In some embodiments, the tissue-specific promoter is a promoter of a gene selected from: neuronal nuclei (NeuN), glial fibrillary acidic protein (GFAP), adenomatous polyposis coli (APC), and ionized calcium-binding adapter molecule 1 (Iba-1). Other appropriate tissue specific promoters will be apparent to the skilled artisan. In some embodiments, the promoter is a chicken Beta-actin promoter.

In some embodiments, the promoter expresses the heterologous nucleic acid in a cell of the CNS. As such, in some embodiments, a therapeutic polypeptide or a therapeutic nucleic acid of the disclosure may be used to treat a tauopathy. In some embodiments, the promoter expresses the heterologous nucleic acid in a brain cell. A brain cell may refer to any brain cell known in the art, including without limitation a neuron (such as a sensory neuron, motor neuron, interneuron, dopaminergic neuron, medium spiny neuron, cholinergic neuron, GABAergic neuron, pyramidal neuron, etc.), a glial cell (such as microglia, macroglia, astrocytes, oligodendrocytes, ependymal cells, radial glia, etc.), a brain parenchyma cell, microglial cell, ependemal cell, and/or a Purkinje cell. In some embodiments, the promoter expresses the heterologous nucleic acid in a neuron and/or glial cell. In some embodiments, the neuron is a medium spiny neuron of the caudate nucleus, a medium spiny neuron of the putamen, a neuron of the cortex layer IV and/or a neuron of the cortex layer V.

Various promoters that express transcripts (e.g., a heterologous transgene) in CNS cells, brain cells, neurons, and glial cells are known in the art and described herein. Such promoters can comprise control sequences normally associated with the selected gene or heterologous control sequences. Often, useful heterologous control sequences include those derived from sequences encoding mammalian or viral genes. Examples include, without limitation, the SV40 early promoter, mouse mammary tumor virus LTR promoter, adenovirus major late promoter (Ad MLP), a herpes simplex virus (HSV) promoter, a cytomegalovirus (CMV) promoter such as the CMV immediate early promoter region (CMVIE), a rous sarcoma virus (RSV) promoter, synthetic promoters, hybrid promoters, and the like. In addition, sequences derived from nonviral genes, such as the murine metallothionein gene, may also be used. Such promoter sequences are commercially available from, e.g., Stratagene (San Diego, CA). CNS-specific promoters and inducible promoters may be used. Examples of CNS-specific promoters include without limitation those isolated from CNS-specific genes such as myelin basic protein (MBP), glial fibrillary acid protein (GFAP), and neuron specific enolase (NSE). Examples of inducible promoters include DNA responsive elements for ecdysone, tetracycline, metallothionein, and hypoxia, inter alia.

The present disclosure contemplates the use of a recombinant viral genome for introduction of one or more nucleic acid sequences encoding for an artificial miRNA as described herein or packaging into an AAV viral particle. The recombinant viral genome may include any element to establish the expression of a miRNA, for example, a promoter, a heterologous nucleic acid, an ITR, a ribosome binding element, terminator, enhancer, selection marker, intron, polyA signal, and/or origin of replication. In some embodiments, the rAAV vector comprises one or more of an enhancer, a splice donor/splice acceptor pair, a matrix attachment site, or a polyadenylation signal.

In some embodiments, the administration of an effective amount of rAAV particles comprising a vector encoding an artificial miRNA transduces cells (e.g., CNS cells, brain cells, neurons, and/or glial cells) at or near the site of administration (e.g., the striatum and/or cortex) or more distal to the site of administration. In some embodiments, more than about any of 5%, 10%, 15%, 20%, 25%, 30%, 35%, 40%, 45%, 50%, 55%, 60%, 65%, 70%, 75% or 100% of neurons are transduced. In some embodiments, about 5% to about 100%, about 10% to about 50%, about 10% to about 30%, about 25% to about 75%, about 25% to about 50%, or about 30% to about 50% of the neurons are transduced. Methods to identify neurons transduced by recombinant viral particles expressing miRNA are known in the art; for example, immunohistochemistry, RNA detection (e.g., qPCR, Northern blotting, RNA-seq, in situ hybridization, and the like) or the use of a co-expressed marker such as enhanced green fluorescent protein can be used to detect expression.

In some aspects, the disclosure provides viral particles comprising a recombinant self-complementing genome (e.g., a self-complementary rAAV vector). AAV viral particles with self-complementing vector genomes and methods of use of self-complementing AAV genomes are described in U.S. Pat. Nos. 6,596,535; 7,125,717; 7,465,583; 7,785,888; 7,790,154; 7,846,729; 8,093,054; and 8,361,457; and Wang Z., et al., (2003) Gene Ther 10:2105-2111, each of which are incorporated herein by reference in its entirety. A rAAV comprising a self-complementing genome will quickly form a double stranded DNA molecule by virtue of its partially complementing sequences (e.g., complementing coding and non-coding strands of a heterologous nucleic acid). In some embodiments, the vector comprises first nucleic acid sequence encoding the heterologous nucleic acid and a second nucleic acid sequence encoding a complement of the nucleic acid, where the first nucleic acid sequence can form intrastrand base pairs with the second nucleic acid sequence along most or all of its length.

In some embodiments, the first heterologous nucleic acid sequence encoding a miRNA and a second heterologous nucleic acid sequence encoding the complement of the miRNA are linked by a mutated ITR (e.g., the right ITR). In some embodiments, the ITR comprises the polynucleotide sequence 5′-CACTCCCTCTCTGCGCGCTCGCTCGCTCACTGAGGCC GGGCGACCAAAGGTCGCCCACGCCCGGGCTTTGCCCGGGCG-3′ (SEQ ID NO: 49).

The mutated ITR comprises a deletion of the D region comprising the terminal resolution sequence. As a result, on replicating an AAV viral genome, the rep proteins will not cleave the viral genome at the mutated ITR and as such, a recombinant viral genome comprising the following in 5′ to 3′ order will be packaged in a viral capsid: an AAV ITR, the first heterologous polynucleotide sequence including regulatory sequences, the mutated AAV ITR, the second heterologous polynucleotide in reverse orientation to the first heterologous polynucleotide and a third AAV ITR.

### V. Viral Particles and Methods of Producing Viral Particles

The disclosure provides, inter alia, recombinant viral particles comprising a nucleic acid encoding an artificial miRNA of the present disclosure, as well as methods of use thereof to treat a disease or disorder in a mammal; e.g., a tauopathy.

### Viral Particles

The disclosure provides viral particles comprising the miRNA molecules as disclosed herein. In some embodiments, the disclosure provides viral particles for delivering the miRNA molecules of the disclosure as disclosed herein. For example, the disclosure provides methods of using recombinant viral particles to deliver miRNA to treat a disease or disorder in a mammal; e.g., rAAV particles comprising miRNA to treat a tauopathy. In some embodiments, the recombinant viral particle is a recombinant AAV particle. In some embodiments, the viral particle is a recombinant AAV particle comprising a nucleic acid comprising a sequence an artificial miRNA of the present disclosure flanked by one or two ITRs. The nucleic acid is encapsidated in the AAV particle. The AAV particle also comprises capsid proteins. In some embodiments, the nucleic acid comprises the coding sequence(s) of interest (e.g., nucleic acid an miRNA of the present disclosure) operatively linked components in the direction of transcription, control sequences including transcription initiation and termination sequences, thereby forming an expression construct. The expression construct is flanked on the 5′ and 3′ end by at least one functional AAV ITR sequences. By “functional AAV ITR sequences” it is meant that the ITR sequences function as intended for the rescue, replication and packaging of the AAV virion. See Davidson et al., PNAS, 2000, 97 (7) 3428-32; Passini et al., J. Virol., 2003, 77 (12): 7034-40; and Pechan et al., Gene Ther., 2009, 16:10-16, all of which are incorporated herein in their entirety by reference. For practicing some aspects of the disclosure, the recombinant vectors comprise at least all of the sequences of AAV essential for encapsidation and the physical structures for infection by the rAAV. AAV ITRs for use in the vectors of the disclosure need not have a wild-type nucleotide sequence (e.g., as described in Kotin, Hum. Gene Ther., 1994, 5:793-801), and may be altered by the insertion, deletion or substitution of nucleotides or the AAV ITRs may be derived from any of several AAV serotypes. More than 40 serotypes of AAV are currently known, and new serotypes and variants of existing serotypes continue to be identified. See Gao et al., PNAS, 2002, 99 (18): 11854-6; Gao et al., PNAS, 2003, 100 (10): 6081-6; and Bossis et al., J. Virol., 2003, 77 (12): 6799-810. Use of any AAV serotype is considered within the scope of the present disclosure. In some embodiments, a rAAV vector is a vector derived from an AAV serotype, including without limitation, AAV ITRs are AAV1, AAV2, AAV3, AAV4, AAV5, AAV6, AAV7, AAV8, AAVrh8, AAVrh8R, AAV9, AAV10, AAVrh10, AAV11, AAV12, AAV2R471A, AAV DJ, a goat AAV, bovine AAV, or mouse AAV capsid serotype or the like. In some embodiments, the nucleic acid in the AAV comprises an ITR of AAV1, AAV2, AAV3, AAV4, AAV5, AAV6, AAV7, AAV8, AAVrh8, AAVrh8R, AAV9, AAV10, AAVrh10, AAV11, AAV12, AAV2R471A, AAV DJ, a goat AAV, bovine AAV, or mouse AAV capsid serotype or the like. In some embodiments, the nucleic acid in the AAV further encodes a miRNA as described herein. In some embodiments the rAAV particle comprise an AAV1, an AAV2HBKO capsid (e.g., as described in WO2015168666), an AAV9 capsid, a PHP.B capsid, a PHP.eB capsid, or an Olig001 capsid.

For example, the nucleic acid in the AAV can comprise at least one ITR of any AAV serotype contemplated herein and can further encode a miRNA comprising a first strand and a second strand, wherein a) the first strand and the second form a duplex; b) the first strand comprises a guide region, and c) the second strand comprises a non-guide region, wherein the non-guide region comprises a two nucleotide deletion at bases 9 and 10 to create bulge in the guide strand. In some embodiments, a vector may include a stuffer nucleic acid. In some embodiments, the stuffer nucleic acid may encode a green fluorescent protein. In some embodiments, the stuffer nucleic acid may be located between the promoter and the nucleic acid encoding the miRNA. In some embodiments, the stuffer nucleic acid is an A1AT stuffer nucleic acid.

Different AAV serotypes are used to optimize transduction of particular target cells or to target specific cell types within a particular target tissue (e.g., a diseased tissue). A rAAV particle can comprise viral proteins and viral nucleic acids of the same serotype or a mixed serotype. For example, in some embodiments a rAAV particle can comprise AAV1 capsid proteins and at least one AAV2 ITR or it can comprise AAV2 capsid proteins and at least one AAV1 ITR. Any combination of AAV serotypes for production of a rAAV particle is provided herein as if each combination had been expressly stated herein. In some embodiments, the disclosure provides rAAV particles comprising an AAV1 capsid and a rAAV vector of the present disclosure (e.g., an expression construct comprising nucleic acid encoding a miRNA of the present disclosure), flanked by at least one AAV2 ITR. In some embodiments, the disclosure provides rAAV particles comprising an AAV2 capsid. In some embodiments the rAAV particle comprise an AAV1, an AAV2HBKO capsid (e.g., as described in WO2015168666), an AAV9 capsid, a PHP.B capsid, a PHP.eB capsid, or an Olig001.

In some aspects, the disclosure provides viral particles comprising a recombinant self-complementing genome. AAV viral particles with self-complementing genomes and methods of use of self-complementing AAV genomes are described in U.S. Pat. Nos. 6,596,535; 7,125,717; 7,465,583; 7,785,888; 7,790,154; 7,846,729; 8,093,054; and 8,361,457; and Wang Z., et al., (2003) Gene Ther 10:2105-2111, each of which are incorporated herein by reference in its entirety. A rAAV comprising a self-complementing genome will quickly form a double stranded DNA molecule by virtue of its partially complementing sequences (e.g., complementing coding and non-coding strands of a transgene). In some embodiments, the disclosure provides an AAV viral particle comprising an AAV genome, wherein the rAAV genome comprises a first heterologous polynucleotide sequence (e.g., a miRNA of the present disclosure) and a second heterologous polynucleotide sequence (e.g., antisense strand of a miRNA of the present disclosure) wherein the first heterologous polynucleotide sequence can form intrastrand base pairs with the second polynucleotide sequence along most or all of its length. In some embodiments, the first heterologous polynucleotide sequence and a second heterologous polynucleotide sequence are linked by a sequence that facilitates intrastrand base pairing; e.g., a hairpin DNA structure. Hairpin structures are known in the art, for example in miRNA or siRNA molecules. In some embodiments, the first heterologous polynucleotide sequence and a second heterologous polynucleotide sequence are linked by a mutated ITR (e.g., the right ITR). In some embodiments, the ITR comprises the polynucleotide sequence 5′-ttggccactccctctctgegcgctcgctegctcactgaggccgcccgggcaaagcccgggcgtcgggcgacctttggtcgcccggcc tcagtgagcgagcgagcgcgcagagagggagtggccaactccatcactaggggttcct-3′ (SEQ ID NO: 50). The mutated ITR comprises a deletion of the D region comprising the terminal resolution sequence. As a result, on replicating an AAV viral genome, the rep proteins will not cleave the viral genome at the mutated ITR and as such, a recombinant viral genome comprising the following in 5′ to 3′ order will be packaged in a viral capsid: an AAV ITR, the first heterologous polynucleotide sequence including regulatory sequences, the mutated AAV ITR, the second heterologous polynucleotide in reverse orientation to the first heterologous polynucleotide and a third AAV ITR. In some embodiments, the disclosure provides AAV viral particles comprising a recombinant viral genome comprising a functional AAV2 ITR, a first polynucleotide sequence encoding a miRNA of the present disclosure, a mutated AAV2 ITR comprising a deletion of the D region and lacking a functional terminal resolution sequence, a second polynucleotide sequence comprising the complementary sequence to the sequence encoding a miRNA of the present disclosure, of the first polynucleotide sequence and a functional AAV2 ITR.

### Production of Viral Particles

rAAV particles can be produced using methods known in the art. See, e.g., U.S. Pat. Nos. 6,566,118; 6,989,264; and 6,995,006. In practicing the disclosure, host cells for producing rAAV particles include mammalian cells, insect cells, plant cells, microorganisms and yeast. Host cells can also be packaging cells in which the AAV rep and cap genes are stably maintained in the host cell or producer cells in which the AAV vector genome is stably maintained. Exemplary packaging and producer cells are derived from 293, A549 or HeLa cells. AAV vectors are purified and formulated using standard techniques known in the art.

Methods known in the art for production of rAAV vectors include but are not limited to transfection, stable cell line production, and infectious hybrid virus production systems which include adenovirus-AAV hybrids, herpesvirus-AAV hybrids (Conway, J E et al., (1997).J. Virology 71 (11): 8780-8789) and baculovirus-AAV hybrids. rAAV production cultures for the production of rAAV virus particles all require; 1) suitable host cells, including, for example, human-derived cell lines such as HeLa, A549, or 293 cells, or insect-derived cell lines such as SF-9, in the case of baculovirus production systems; 2) suitable helper virus function, provided by wild-type or mutant adenovirus (such as temperature sensitive adenovirus), herpes virus, baculovirus, or a plasmid construct providing helper functions; 3) AAV rep and cap genes and gene products; 4) a nucleic acid (such as a therapeutic nucleic acid) flanked by at least one AAV ITR sequences; and 5) suitable media and media components to support rAAV production. In some embodiments, the AAV rep and cap gene products may be from any AAV serotype. In general, but not obligatory, the AAV rep gene product is of the same serotype as the ITRs of the rAAV vector genome as long as the rep gene products may function to replicated and package the rAAV genome. Suitable media known in the art may be used for the production of rAAV vectors. These media include, without limitation, media produced by Hyclone Laboratories and JRH including Modified Eagle Medium (MEM), Dulbecco's Modified Eagle Medium (DMEM), custom formulations such as those described in U.S. Pat. No. 6,566,118, and Sf-900 II SFM media as described in U.S. Pat. No. 6,723,551, each of which is incorporated herein by reference in its entirety, particularly with respect to custom media formulations for use in production of recombinant AAV vectors. In some embodiments, the AAV helper functions are provided by adenovirus or HSV. In some embodiments, the AAV helper functions are provided by baculovirus and the host cell is an insect cell (e.g., Spodoptera frugiperda (Sf9) cells).

In some embodiments, rAAV particles may be produced by a triple transfection method, such as the exemplary triple transfection method provided infra. Briefly, a plasmid containing a rep gene and a capsid gene, along with a helper adenoviral plasmid, may be transfected (e.g., using the calcium phosphate method) into a cell line (e.g., HEK-293 cells), and virus may be collected and optionally purified. As such, in some embodiments, the rAAV particle was produced by triple transfection of a nucleic acid encoding the rAAV vector, a nucleic acid encoding AAV rep and cap, and a nucleic acid encoding AAV helper virus functions into a host cell, wherein the transfection of the nucleic acids to the host cells generates a host cell capable of producing rAAV particles.

In some embodiments, rAAV particles may be produced by a producer cell line method, such as the exemplary producer cell line method provided infra (see also (referenced in Martin et al., (2013) Human Gene Therapy Methods 24:253-269). Briefly, a cell line (e.g., a HeLa cell line) may be stably transfected with a plasmid containing a rep gene, a capsid gene, and a promoter-heterologous nucleic acid sequence. Cell lines may be screened to select a lead clone for rAAV production, which may then be expanded to a production bioreactor and infected with an adenovirus (e.g., a wild-type adenovirus) as helper to initiate rAAV production. Virus may subsequently be harvested, adenovirus may be inactivated (e.g., by heat) and/or removed, and the rAAV particles may be purified. As such, in some embodiments, the rAAV particle was produced by a producer cell line comprising one or more of nucleic acid encoding the rAAV vector, a nucleic acid encoding AAV rep and cap, and a nucleic acid encoding AAV helper virus functions.

In some aspects, a method is provided for producing any rAAV particle as disclosed herein comprising (a) culturing a host cell under a condition that rAAV particles are produced, wherein the host cell comprises (i) one or more AAV package genes, wherein each said AAV packaging gene encodes an AAV replication and/or encapsidation protein; (ii) an rAAV pro-vector comprising a nucleic acid encoding miRNA of the present disclosure as described herein flanked by at least one AAV ITR, and (iii) an AAV helper function; and (b) recovering the rAAV particles produced by the host cell. In some embodiments, said at least one AAV ITR is selected from the group consisting of AAV ITRs are AAV1, AAV2, AAV3, AAV4, AAV5, AAV6, AAV7, AAV8, AAVrh8, AAVrh8R, AAV9, AAV10, AAVrh10, AAV11, AAV12, AAV2R471A, AAV DJ, a goat AAV, bovine AAV, or mouse AAV capsid serotype ITRs or the like. In some embodiments, said encapsidation protein is selected from the group consisting of AAV1, AAV2, AAV3, AAV4, AAV5, AAV6 (e.g., a wild-type AAV6 capsid, or a variant AAV6 capsid such as ShH10, as described in U.S. PG Pub. 2012/0164106), AAV7, AAV8, AAVrh8, AAVrh8R, AAV9 (e.g., a wild-type AAV9 capsid, or a modified AAV9 capsid as described in U.S. PG Pub. 2013/0323226), AAV10, AAVrh10, AAV11, AAV12, a tyrosine capsid mutant, a heparin binding capsid mutant, an AAV2R471A capsid, an AAVAAV2/2-7m8 capsid, an AAV DJ capsid (e.g., an AAV-DJ/8 capsid, an AAV-DJ/9 capsid, or any other of the capsids described in U.S. PG Pub. 2012/0066783), AAV2 N587A capsid, AAV2 E548A capsid, AAV2 N708A capsid, AAV V708K capsid, goat AAV capsid, AAV1/AAV2 chimeric capsid, bovine AAV capsid, mouse AAV capsid, rAAV2/HBoV1 capsid, or an AAV capsid described in U.S. Pat. No. 8,283,151 or International Publication No. WO/2003/042397. In some embodiments, the AAV capsid is an AAV2HBKO capsid as described in WO2015168666. In some embodiments, the AAV capsid is an AAV9 capsid. In some embodiments, the AAV capsid is a PHP.B, PHP.eB or an Olig001 capsid. In some embodiments, a mutant capsid protein maintains the ability to form an AAV capsid. In some embodiments, the encapsidation protein is an AAV5 tyrosine mutant capsid protein. In further embodiments, the rAAV particle comprises capsid proteins of an AAV serotype from Clades A-F. In some embodiments, the rAAV particles comprise an AAV1 capsid and a recombinant genome comprising AAV2 ITRs and nucleic acid encoding a miRNA of the present disclosure. In a further embodiment, the rAAV particles are purified. The term “purified” as used herein includes a preparation of rAAV particles devoid of at least some of the other components that may also be present where the rAAV particles naturally occur or are initially prepared from. Thus, for example, isolated rAAV particles may be prepared using a purification technique to enrich it from a source mixture, such as a culture lysate or production culture supernatant. Enrichment can be measured in a variety of ways, such as, for example, by the proportion of DNase-resistant particles (DRPs) or genome copies (gc) present in a solution, or by infectivity, or it can be measured in relation to a second, potentially interfering substance present in the source mixture, such as contaminants, including production culture contaminants or in-process contaminants, including helper virus, media components, and the like.

Numerous methods are known in the art for production of adenoviral vector particles. For example, for a gutted adenoviral vector, the adenoviral vector genome and a helper adenovirus genome may be transfected into a packaging cell line (e.g., a 293 cell line). In some embodiments, the helper adenovirus genome may contain recombination sites flanking its packaging signal, and both genomes may be transfected into a packaging cell line that expresses a recombinase (e.g., the Cre/loxP system may be used), such that the adenoviral vector of interest is packaged more efficiently than the helper adenovirus (see, e.g., Alba, R. et al. (2005) Gene Ther. 12 Suppl 1: S18-27). Adenoviral vectors may be harvested and purified using standard methods, such as those described herein.

Numerous methods are known in the art for production of lentiviral vector particles. For example, for a third-generation lentiviral vector, a vector containing the lentiviral genome of interest with gag and pol genes may be co-transfected into a packaging cell line (e.g., a 293 cell line) along with a vector containing a rev gene. The lentiviral genome of interest also contains a chimeric LTR that promotes transcription in the absence of Tat (see Dull, T. et al. (1998).J. Virol. 72:8463-71). Lentiviral vectors may be harvested and purified using methods (e.g., Segura M M, et al., (2013) Expert Opin Biol Ther. 13 (7): 987-1011) described herein.

Numerous methods are known in the art for production of HSV particles. HSV vectors may be harvested and purified using standard methods, such as those described herein. For example, for a replication-defective HSV vector, an HSV genome of interest that lacks all of the immediate early (IE) genes may be transfected into a complementing cell line that provides genes required for virus production, such as ICP4, ICP27, and ICPO (see, e.g., Samaniego, L. A. et al. (1998) J. Virol. 72:3307-20). HSV vectors may be harvested and purified using methods described (e.g., Goins, W F et al., (2014) Herpes Simplex Virus Methods in Molecular Biology 1144:63-79).

Also provided herein are pharmaceutical compositions comprising a recombinant viral particle comprising a transgene encoding a miRNA of the present disclosure and a pharmaceutically acceptable carrier. The pharmaceutical compositions may be suitable for any mode of administration described herein. A pharmaceutical composition of a recombinant viral particle comprising a nucleic acid encoding a miRNA of the present disclosure can be introduced to the brain. For example, a recombinant viral particle comprising a nucleic acid encoding a miRNA of the present disclosure can be administered intrastriatally. Any of the recombinant viral particles of the present disclosure may be used, including rAAV, adenoviral, lentiviral, and HSV particles.

In some embodiments, the pharmaceutical compositions comprising a recombinant viral particle comprising a transgene encoding a miRNA of the present disclosure described herein and a pharmaceutically acceptable carrier is suitable for administration to human. Such carriers are well known in the art (see, e.g., Remington's Pharmaceutical Sciences, 15^{th} Edition, pp. 1035-1038 and 1570-1580). In some embodiments, the pharmaceutical compositions comprising a rAAV described herein and a pharmaceutically acceptable carrier is suitable for injection into the brain of a mammal (e.g., intrastriatal administration). In some embodiments, the pharmaceutical compositions comprising a recombinant lentiviral particle described herein and a pharmaceutically acceptable carrier is suitable for injection into the brain of a mammal (e.g., intrastriatal administration). In some embodiments, the pharmaceutical compositions comprising a recombinant adenoviral particle described herein and a pharmaceutically acceptable carrier is suitable for injection into the brain of a mammal (e.g., intrastriatal administration). In some embodiments, the pharmaceutical compositions comprising a recombinant HSV particle described herein and a pharmaceutically acceptable carrier is suitable for injection into the brain of a mammal (e.g., intrastriatal administration).

Such pharmaceutically acceptable carriers can be sterile liquids, such as water and oil, including those of petroleum, animal, vegetable or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. The pharmaceutical composition may further comprise additional ingredients, for example preservatives, buffers, tonicity agents, antioxidants and stabilizers, nonionic wetting or clarifying agents, viscosity-increasing agents, and the like. The pharmaceutical compositions described herein can be packaged in single unit dosages or in multidosage forms. The compositions are generally formulated as sterile and substantially isotonic solution.

### VI. Methods of Treatment

Certain aspects of the present disclosure relate to methods of treating a tauopathy by reducing levels of tau protein in an individual in need thereof. In some embodiments, the invention provides methods of treating a tauopathy by administering an effective amount of an expression cassette (e.g., an expression cassette delivered in a rAAV particle) for expressing an artificial miRNA of the present disclosure. Examples of tauopathies include, but are not limited to supranuclear palsy, Alzheimer's disease, corticobasal degeneration, chronic traumatic encephalopathy, Pick disease, and post-encephalitic parkinsonism.

The expression cassette (e.g., expression cassette delivered in a rAAV particle) for expressing the artificial miRNA may be administered through various routes. In some embodiments, the administration includes direct spinal cord injection and/or intracerebral administration. In some embodiments, the administration is at a site selected from the cerebrum, medulla, pons, cerebellum, intracranial cavity, meninges surrounding the brain, dura mater, arachnoid mater, pia mater, cerebrospinal fluid (CSF) of the subarachnoid space surrounding the brain, deep cerebellar nuclei of the cerebellum, ventricular system of the cerebrum, subarachnoid space, striatum, cortex, septum, thalamus, hypothalamus, and the parenchyma of the brain. In some embodiments, the administration comprises intracerebroventricular injection into at least one cerebral lateral ventricle. In some embodiments, the administration comprises intrathecal injection in the cervical, thoracic, and/or lumbar region. In some embodiments, the administration comprises intrastriatal injection. In some embodiments, the administration comprises intrathalamic injection.

In some embodiments, routes of administration to the central nervous system may comprise an intraparenchymal route. In some embodiments, the intraparenchymal route may comprise thalamic, striatal, or hippocampal. In some embodiments, routes of administration to the central nervous system may comprise an intra-cerebral spinal fluid (CSF) route. In some embodiments, the intra-CSF route may comprise intracerebroventricular, intra-cisternal magna, or intrathecal. In some embodiments, routes of administration to the central nervous system may comprise a peripheral route. In some embodiments, the peripheral route may comprise an intravenous route. In some embodiments, routes of administration to the central nervous system may comprise an experimental route. In some embodiments, the experimental route may comprise an intranasal route.

In some embodiments, a route of administration (ROA) may comprise an intra-cerebral spinal fluid (intra-CSF) ROA. In some embodiments, the intra-CSF ROA may comprise intracerebroventricular (ICV), intra-cisternal magna (ICM), or intrathecal (IT) ROA.

An effective amount of rAAV (in some embodiments in the form of particles) is administered, depending on the objectives of treatment. For example, where a low percentage of transduction can achieve the desired therapeutic effect, then the objective of treatment is generally to meet or exceed this level of transduction. In some instances, this level of transduction can be achieved by transduction of only about 1 to 5% of the target cells of the desired tissue type, in some embodiments at least about 20% of the cells of the desired tissue type, in some embodiments at least about 50%, in some embodiments at least about 80%, in some embodiments at least about 95%, in some embodiments at least about 99% of the cells of the desired tissue type. The rAAV composition may be administered by one or more administrations, either during the same procedure or spaced apart by days, weeks, months, or years. One or more of any of the routes of administration described herein may be used. In some embodiments, multiple vectors may be used to treat the human.

In some embodiments of the above aspects, the rAAV is administered via direct injection into the spinal cord, via intrathecal injection, or via intracisternal injection. In some embodiments, the rAAV is administered to more than one location of the spinal cord or cisterna magna. In some embodiments, the rAAV is administered to more than one location of the spinal cord. In some embodiments, the rAAV is administered to one or more of a lumbar subarachnoid space, thoracic subarachnoid space and a cervical subarachnoid space of the spinal cord. In some embodiments, the rAAV is administered to the cisterna magna.

In some embodiments, the invention provides a method for treating a human with a tauopathy by administering an effective amount of a pharmaceutical composition comprising a recombinant viral vector encoding an artificial miRNA of the present disclosure. In some embodiments, the pharmaceutical composition comprises one or more pharmaceutically acceptable excipients.

In some embodiments, the methods comprise administering an effective amount of a pharmaceutical composition comprising a recombinant viral vector encoding an artificial miRNA polypeptide of the present disclosure to a tauopathy in an individual in need thereof. In some embodiments, the viral titer of the viral particles (e.g., rAAV particles) is at least about any of 5×10¹², 6×10¹², 7×10¹², 8×10¹², 9×10¹², 10×10¹², 11×10¹², 15×10¹², 20×10¹², 25×10¹², 30×10¹², or 50×10¹² genome copies/mL. In some embodiments, the viral titer of the viral particles (e.g., rAAV particles) is about any of 5×10¹² to 6×10¹², 6×10¹² to 7 ×10¹², 7×10¹² to 8×10¹², 8×10¹² to 9×10¹², 9×10¹² to 10×10¹², 10×10¹² to 11×10¹², 11×10¹² to 15×10¹², 15×10¹² to 20×10¹², 20×10¹² to 25×10¹², 25×10¹² to 30×10¹², 30 ×10¹² to 50×10¹², or 50×10¹² to 100×10¹² genome copies/mL. In some embodiments, the viral titer of the viral particles (e.g., rAAV particles) is about any of 5×10¹² to 10×10¹², 10×10¹² to 25×10¹², or 25×10¹² to 50×10¹² genome copies/mL. In some embodiments, the viral titer of the viral particles (e.g., rAAV particles) is at least about any of 5×10⁹, 6×10⁹, 7×10⁹, 8×10⁹, 9×10⁹, 10×10⁹, 11×10⁹, 15×10⁹, 20×10⁹, 25×10⁹, 30 ×10⁹, or 50×10⁹ transducing units/mL. In some embodiments, the viral titer of the viral particles (e.g., rAAV particles) is about any of 5×10⁹ to 6 ×10⁹, 6 ×10⁹ to 7×10⁹, 7×10⁹ to 8×10⁹, 8×10⁹ to 9×10⁹, 9×10⁹ to 10×10⁹, 10×10⁹ to 11×10⁹, 11×10⁹ to 15×10⁹, 15×10⁹ to 20×10⁹, 20×10⁹ to 25×10⁹, 25×10⁹ to 30×10⁹, 30×10⁹ to 50×10⁹ or 50×10⁹ to 100×10⁹ transducing units/mL. In some embodiments, the viral titer of the viral particles (e.g., rAAV particles) is about any of 5×10⁹ to 10×10⁹, 10 ×10⁹ to 15×10⁹, 15×10⁹ to 25×10⁹, or 25×10⁹ to 50×10⁹ transducing units/mL. In some embodiments, the viral titer of the viral particles (e.g., rAAV particles) is at least any of about 5×10¹⁰, 6×10¹⁰, 7×10¹⁰, 8×10¹⁰, 9×10¹⁰, 10×10¹⁰, 11×10¹⁰, 15×10¹⁰, 20×10¹⁰, 25×10¹⁰ 30×10¹⁰, 40 ×10¹⁰, or 50×10¹⁰ infectious units/mL. In some embodiments, the viral titer of the viral particles (e.g., rAAV particles) is at least any of about 5×10¹⁰ to 6×10¹⁰, 6×10¹⁰ to 7 ×10¹⁰, 7×10¹⁰ to 8×10¹⁰, 8×10¹⁰ to 9×10¹⁰, 9×10¹⁰ to 10 ×10¹⁰, 10 ×10¹⁰ to 11×10¹⁰, 11 ×10¹⁰ to 15×10¹⁰, 15×10¹⁰ to 20×10¹⁰, 20×10¹⁰ to 25×10¹⁰, 25×10¹⁰ to 30×10¹⁰, 30 ×10¹⁰ to 40×10¹⁰, 40×10¹⁰ to 50×10¹⁰, or 50×10¹⁰ to 100×10¹⁰ infectious units/mL. In some embodiments, the viral titer of the viral particles (e.g., rAAV particles) is at least any of about 5×10¹⁰ to 10×10¹⁰, 10×10¹⁰ to 15×10¹⁰, 15×10¹⁰ to 25×10¹⁰, or 25×10¹⁰ to 50×10¹⁰ infectious units/mL. In some embodiments, the viral particles are rAAV particles.

In some embodiments, the dose of viral particles administered to the individual is at least about any of 1×10⁸ to about 6×10¹³ genome copies/kg of body weight. In some embodiments, the dose of viral particles administered to the individual is about any of 1×10⁸ to about 6×10¹³ genome copies/kg of body weight. In some embodiments, the dose of viral particles administered to the individual is about any of 1×10¹⁰, 2×10¹⁰, 3×10¹⁰, 4×10¹⁰, 5×10¹⁰, 6 ×10¹⁰, 7×10¹⁰, 8×10¹⁰, 9×10¹⁰, 1 ×10¹¹, 2×10¹¹, 3×10¹¹, 4×10¹¹, 5×10¹¹, 6×10¹¹, 7×10¹¹, 8×10¹¹, 9×10¹¹, 1 ×10¹², 2×10¹², 13×10¹², 4×10¹², 5×10¹², 6×10¹², 7×10¹², 8×10¹², 9×10¹², or 1×10¹³ genome copies/kg of body weight.

In some embodiments, the total amount of viral particles administered to the individual is at least about any of 1×10⁹ to about 1×10¹⁴ genome copies. In some embodiments, the total amount of viral particles administered to the individual is about any of 1 ×10⁹ to about 1×10¹⁴ genome copies. In some embodiments, the total amount of viral particles administered to the individual is about any of 1×10¹¹, 2×10¹¹, 3×10¹¹, 4×10¹¹, 5×10¹¹, 6×10¹¹, 7 ×10¹¹, 8×10¹¹, 9×10¹¹, 1 ×10¹², 2×10¹², 3×10¹², 4×10¹², 5×10¹², 6×10¹², 7×10¹², 8×10¹², 9×10¹², 1×10¹³, 2×10¹³, 13×10¹³, 4×10¹³, 5×10¹³, 6×10¹³, 7×10¹³, 8×10¹³, 9×10¹³, or 1×10¹⁴ genome copies.

In some embodiments, Tau22 mouse may be used to measure the durability of MAPT knockdown. In some embodiments, Tau22 mouse may be used to evaluate the impact of tau reduction on aggregate formation and/or associated neurodegeneration. In some embodiments, Tau01 and Tau12 may also be administered to nonhuman primates to confirm tau knockdown and/or evaluate fluid biomarkers of target engagement in a large animal model.

In some embodiments, in silico design tools may be used to identify artificial miRNA (amiRNA) sequences that may comprise low off-targeting potential, homology across species, or a combination thereof. In some embodiments, sequences may be tested for MAPT knockdown efficiency in vitro using U2OS cells, wherein the U2OS cells may stably express 4R human tau. In some embodiments, tau protein may be quantified by ELISA. In some embodiments, tau protein may be quantified by ELISA, three days post-transfection. In some embodiments, top candidates may be cloned into AAV vectors to evaluate MAPT knockdown and/or efficacy readouts in the Tau22 mouse model of tauopathy. In some embodiments, the mice may overexpress 1N4R human tau with two FTD-associated mutations that may drive progressive neuronal accumulation of tau aggregates and/or subsequent neurodegeneration.

### VII. Articles of Manufacture and Kits

Also provided are kits or articles of manufacture for use in the methods described herein. In aspects, the kits comprise the compositions described herein (e.g., a recombinant viral particle of the present disclosure, such as a rAAV particle comprising nucleic acid encoding a miRNA of the present disclosure) in suitable packaging. Suitable packaging for compositions (such as intrastriatal compositions) described herein are known in the art, and include, for example, vials (such as sealed vials), vessels, ampules, bottles, jars, flexible packaging (e.g., sealed Mylar or plastic bags), and the like. These articles of manufacture may further be sterilized and/or sealed.

The present disclosure also provides kits comprising compositions described herein and may further comprise instruction(s) on methods of using the composition, such as uses described herein. The kits described herein may further include other materials desirable from a commercial and user standpoint, including other buffers, diluents, filters, needles, syringes, and package inserts with instructions for performing any methods described herein. For example, in some embodiments, the kit comprises a composition of recombinant viral particles comprising a transgene encoding a miRNA of the present disclosure for delivery of at least 1×10⁹ genome copies into the brain of a mammal (e.g., through intrastriatal administration) to a primate as described herein, a pharmaceutically acceptable carrier suitable for injection into the brain of a primate, and one or more of: a buffer, a diluent, a filter, a needle, a syringe, and a package insert with instructions for performing injections into the brain of a primate (e.g., intrastriatal administration). In some embodiments, the kit comprising instructions for treating a neurodegenerative synucleinopathy with the recombinant viral particles described herein. In some embodiments, the kit comprising instructions for using the recombinant viral particles described herein according to any one of the methods described herein

### EXAMPLES

The disclosure will be more fully understood by reference to the following examples. They should not, however, be construed as limiting the scope of the disclosure. It is understood that the examples and embodiments described herein are for illustrative purposes only and that various modifications or changes in light thereof will be suggested to persons skilled in the art and are to be included within the spirit and purview of this application and scope of the appended claims.

### General Methods

### Plasmids, ITR Vectors and AAV Generation

In vitro MAPT or control artificial miRNAs were expressed as mir155-embedded hairpins under the control of the human CMV enhancer/EF1alpha promoter. Sequences were designed to match regions homologous only to human/NHP MAPT. The control artificial miRNA encodes a non-targeting artificial miRNA sequence designed for minimal seed-mediated off-target gene repression. To generate recombinant AAV serotype vectors encoding artificial miRNAs, the artificial miRNA cassettes were cloned into a plasmid containing AAV2 inverted terminal repeats (ITRs) under control of the human cytomegalovirus enhancer/chicken beta-actin (CBA) promoter. To generate AAV, HEK293 cells were transfected using PEI (polyethyleneimine) with a 1:1:1 ratio of three plasmids (containing the ITR, AAV rep/cap and Ad helper). The Ad helper plasmid (pHelper) was obtained from Stratagene/Agilent Technologies (Santa Clara, CA). AAV purification was performed using cesium chloride ultracentrifugation, and virus was titered using qPCR against the poly A sequence.

### U20S Cell Culture and Transfection

U2OS cells stably expressing human 1N4R tau with the G272V and P301S mutations were maintained in DMEM+10% FCS with 100 ug/mL Hygromycin B (Invitrogen 10687010). Transfection of artificial miRNA plasmid DNA was performed using Lipofectamine 3000 (Life Technologies) following the manufacturer's instructions. Cells were lysed in 1% Triton X-100 (Sigma) in PBS with protease inhibitor cocktail (Roche) three days later for use in the anti-tau ELISA assay.

### Human Tau (HT7 Antibody) Sandwich ELISA

Immulon IIHB 96 well plates (Thermo Scientific) were coated with mouse-anti-human tau (HT7 clone, Thermo Scientific MN10008) at 2 ug/mL in PBS, overnight at room temperature. Plates were then washed, blocked in PBS+0.1% Tween 20 (PBST)+1.5% BSA before incubation with cell lysates. Wells were washed in PBST, then incubated with rabbit anti-Tau (DAKO A0027) for 1.5 hours, following by washing in PBST and incubation with HRP-conjugated donkey-anti-rabbit (Jackson Immuno 711-035-152). Plates were washed in PBST, developed with TMB substrate (SeraCare), quenched with 0.5M H2SO4, and levels of tau quantified via absorbance at 450 nm using a Spectramax M5 plate reader (Molecular Devices). Values were normalized to control artificial miRNA-treated samples to assess relative tau knockdown.

### Animal Use and Care

All procedures were performed according to a protocol approved by the Institutional Animal Care and Use Committee (IACUC) at Sanofi, as per guidelines specified by the Guide for the Care and Use of Laboratory Animals, NIH. Adult Thy-Tau22 (Schindowski et al., 2006) animals were group housed except in stereotactic surgical studies, in which they were housed singly to enable proper recovery. Mice were maintained on a 12 hour light/dark cycle with food and water available ad libitum. Each animal was identified with a unique ear tag.

Stereotaxic Injections with AAV-Artificial miRNA Vectors

Surgery was performed according to procedures approved by IACUC at Sanofi. Mice were anaesthetized by isofluorane exposure and secured on a stereotactic frame (Stoelting) with constant isoflurane perfusion. The scalp was shaved and an incision was made down the midline to locate bregma. A small burr hole was drilled above the desired location within the brain. A Hamilton syringe was mounted onto a microcontrolled stereotactic frame (Stoelting), and the needle was slowly lowered to the appropriate depth. For striatal injections, 3E10 vector genomes were injected into each of two injection sites with coordinates AP+0.5, DV-3.0, and ML+/−2.2. For hippocampal injections, 7.5E9 vector genomes were injected into each of two injection sites with coordinates AP-2.5, DV-2.0, and ML+/−1.5. In each surgery, virus was injected at a rate of 0.5 μL per minute. The needle was left in for two more minutes to prevent flow of vector back through the needle tract, and then slowly raised out of the brain. Mice were kept warm following surgery and observed continuously until recovery. Four weeks post-injection, mice were euthanized by anesthetic overdose with >150 mg/kg sodium pentobarbital. Following overdose, mice were kept warm until cardiac perfusion with ice-cold PBS.

Striatal Tissue Processing and MAPT mRNA Quantification: By RT-qPCR

Following perfusion, striata from Tau22 mice were dissected, flash frozen in liquid nitrogen, and stored at −80° C.. Tissue was bead homogenized at 4C in TRIzol reagent (Invitrogen) using the TissueLyser II (QIAGEN) for 3 minutes at 30 Hz, followed by aqueous phase separation according to manufacturer's instructions. RNA was isolated from the aqueous phase using the miRNeasy Mini Kit (QIAGEN) according to manufacturer's instructions with on-column DNase treatment using the RNase-free DNase Set (QIAGEN). RNA concentration and purity were assessed by measuring absorbance at A260/280 on a NanoDrop spectrophotometer (Thermo Scientific). mRNA expression was quantified by qPCR using the TaqMan Fast Virus 1-Step Master Mix (Applied Biosystems) with pre-validated Taqman probes targeting human MAPT (Hs00902194_m1) and mouse Ppia (Mm 02342430_g1) (Applied Biosystems) on the Quantstudio 6 (Applied Biosystems). Samples were run in triplicate wells on the same plate with 100 nanograms RNA input per well. Relative levels of human MAPT mRNA were quantified in the MAPT artificial miRNA-treated animals versus control artificial miRNA-treated animals by the 2\hat{ }ΔΔCt method with Ppia expression as housekeeping control using VIAA7 software (VIAA7, Applied Biosystems).

Striatal Tissue Homogenization Following AAV.SAN006-Artificial miRNA Treatment

Following perfusion, striata from Tau22 mice were dissected, flash frozen in liquid nitrogen, and stored at −80° C.. Tissue was bead homogenized at 4C in 1× Tris-EDTA (TE) buffer (Fisher BP2475500) using the TissueLyser II (QIAGEN) for 3 minutes at 30 Hz, aliquoted, and stored at −80° C. until further use.

Striatal Human MAPT mRNA Quantification by RT-dPOR

TE homogenates were thawed on ice and QIAZOL lysis reagent (QIAGEN) was added, followed by phase separation according to manufacturer's instructions. RNA was isolated from the aqueous phase using the miRNeasy 96 Mini Kit (QIAGEN) on the QIAcube HT. RNA concentration and purity were assessed by measuring absorbance at A260/280 on a NanoDrop spectrophotometer (Thermo Scientific). mRNA expression was quantified by reverse transcriptase digital PCR using the QIAcuity One-Step Viral RT-PCR Kit (QIAGEN) with probes targeting human MAPT (Hs00902194_ml; Applied Biosystems) and mouse Hprt1 (Mm.PT.58.29815602; IDT). Reactions were performed using the QIAcuity 8 digital PCR system (QIAGEN). MAPT mRNA was quantified with Hprt1 expression as a housekeeping control using QIAcuity Software Suite (QIAGEN), and expression in the MAPT artificial miRNA-treated animals was normalized to control artificial miRNA-treated animals.

### Hippocampal Tau Protein Detection: Immunofluorescence

Following perfusion, brains were removed and divided down the midline into two halves, termed hemibrains. For each mouse, one hemibrain was post-fixed in 10% neutral buffered formalin (NBF) and paraffin embedded for immunofluorescence. Five μm-thick sagittal sections of brain tissue were cut with a microtome and mounted directly onto glass slides. Sections were immunostained with the indicated antibodies using an automated slide staining system (BOND RX, Leica). Briefly, following antigen retrieval in citrate buffer (ER1, Leica) at 95C for 10 minutes, all steps were performed at room temperature. Sections were blocked for 30 minutes with 5% goat serum, incubated with primary antibodies for 1.5 hrs, and Alexafluor fluorescent secondaries for one hour, washing three times between steps. Antibodies were diluted in PBST (PBS+0.5% Tween-20). Slides were mounted in DAPI-containing medium (ProLong Gold anti-fade with DAPI, Life Technologies). The following primary antibodies were used: human tau (clone Tau13, Covance), NeuN (Millipore ABN78).

Images were acquired on a Zeiss AzioZI epifluorescence microscope at 20× magnification (Plan-Apochromat 20×/0.8). For presentation purposes, images were imported into Adobe Photoshop (2019) for cropping and pseudocoloring into RGB color.

### Small RNA Sequencing

Striatal RNA was isolated from Tau22 animals as described above. Small RNA (<200 bp) libraries were prepared and sequenced using the NEBNext Small RNA Library Prep Set from Illumina. Raw data were filtered and aligned to the Mouse.B38 genome with miRbase.R22 gene model with custom genomes added for each artificial miRNA treatment vector. No mismatches were allowed between the reads and reference sequence, and a custom python script was used to export the aligned mature miRNA sequences and counts.

Retroorbital Injections with AAV-PHP.eB-Artificial miRNA Vectors

Injections were performed according to procedures approved by IACUC at Sanofi. Adult mice were anaesthetized by isofluorane exposure and 40 μL of virus containing 3.9E11 vector genomes were injected into each retroorbital sinus for a total dose of 7.8E11 vector genomes per animal. Four weeks post-injection, mice were euthanized by anesthetic overdose with >150 mg/kg sodium pentobarbital. Following overdose, mice were kept warm until cardiac perfusion with ice-cold PBS.

### Hindbrain Tissue Homogenization

Following perfusion, hindbrains from Tau22 mice were dissected, flash frozen in liquid nitrogen, and stored at −80° C.. Tissue was bead homogenized at 4C in 1× Tris-EDTA (TE) buffer (Fisher BP2475500) using the TissueLyser II (QIAGEN) for 3 minutes at 30 Hz, aliquoted, and stored at −80° C. until further use.

Hindbrain Human MAPT mRNA Quantification by ddPOR

TE homogenates were thawed on ice and QIAZOL lysis reagent (QIAGEN) was added, followed by phase separation according to manufacturer's instructions. RNA was isolated from the aqueous phase using the miRNeasy 96 Advanced Mini Kit (QIAGEN) on the QIAcube HT with on-column DNase treatment using the RNase-free DNase Set (QIAGEN). RNA concentration and purity were assessed by measuring absorbance at A260/280 on a NanoDrop spectrophotometer (Thermo Scientific). mRNA expression was quantified by reverse transcriptase digital droplet PCR using the Bio-Rad 1-Step RT-ddPCR Advanced Kit for Probes with probes targeting human MAPT (Hs00902194_m1; Applied Biosystems) and mouse Hprt1 (Mm.PT.58.29815602; IDT). Reactions were performed using the QX200 AutoDG Droplet Digital PCR System with C1000 Touch 96-well Thermal Cycler (Bio-Rad). MAPT mRNA was quantified with Hprt1 expression as a housekeeping control using Quantasoft Analysis Pro software (Bio-Rad), and expression in the MAPT artificial miRNA-treated animals was normalized to control artificial miRNA-treated animals.

### Hindbrain Human Tau Protein Quantification by ELISA

TE homogenates were thawed on ice and 2× RIPA lysis buffer (Boston BioProducts) was added to a final concentration of 1× with added 1× Halt Protease Inhibitor Cocktail (Thermo Scientific). Samples were incubated on ice for 30 minutes and centrifuged at 14,000 RCF for 15 minutes at 4C. Soluble protein concentration was quantified in the supernatant using DC Protein Assay Kit (Bio-Rad) according to manufacturer's instructions, and RIPA-soluble fractions were diluted to a final concentration of 100 pg/mL Total human tau was quantified by ELISA (Invitrogen KHB0041) according to manufacturer's instructions using a Flexstation 3 plate reader (Molecular Devices), and values were normalized to total protein content.

pTau Immunohistochemistry

Mouse hemibrains were dissected and fixed overnight in 10% neutral-buffered formalin prior to paraffin embedding. Sagittal sections were sliced at 5 μm thickness and immunohistochemistry was performed using the AT8 antibody recognizing phosphorylated tau (Ser202, Thr205) (Invitrogen MN1020) on the LeicaBondRx (Leica Biosystems). Slides were then coverslipped and imaged using a 20× objective on the Aperio AT2 Scanner (Leica Biosystems). Images were analyzed using Harmony software using pixel-based thresholds for weak, moderate, and strong AT8+ signal.

### Quanterix Simoa Assays

Blood samples were collected in K2EDTA tubes at pre-dosing and necropsy timepoints, centrifuged at 4C for 10 minutes at 12,000 RPM, and plasma was collected from the supernatant. At the terminal timepoint, cerebrospinal fluid (CSF) was collected at the terminal timepoint by cisternae magna puncture, and terminal tissue homogenates were prepared as described above. Concentration of tau in tissue homogenates and neurofilament light (NfL) in CSF and plasma were measured by ultrasensitive single-molecule array using the N4PB Simoa assay kit (Quanterix) on the Simoa HD-X analyzer (Quanterix). Concentration of pTau181 in tissue homogenates was measured by ultrasensitive single-molecule array using the pTau181 Simoa assay kit (Quanterix) on the Simoa HD-X analyzer (Quanterix).

### Example 1: Artificial miRNA Sequences Reduce Tau Expression in Human Cells

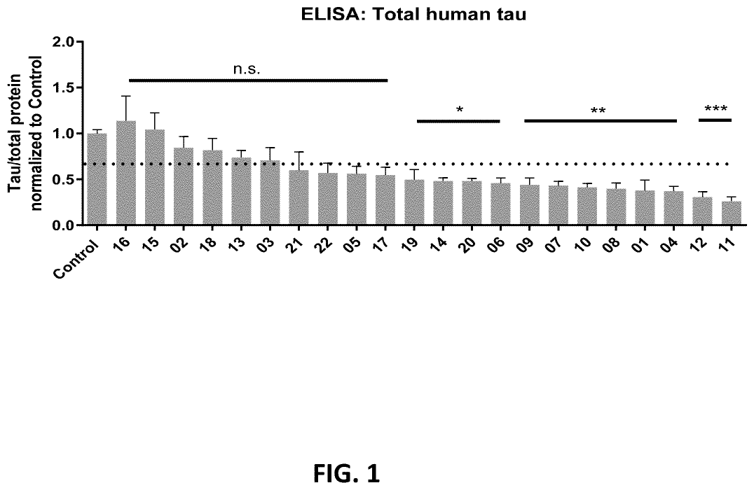

Artificial miRNA (artificial miRNA) sequences were designed targeting human MAPT mRNA. A total of 22 sequences were selected based on: 1) their low potential for off-targeting calculated using siSPOTR (Boudreau et al, 2013); 2) avoidance of known pathogenic MAPT mutations and high frequency single nucleotide polymorphisms (SNP); and 3) high sequence homology between human and nonhuman primates to facilitate translatability. Sequences were each embedded within the murine miR 155 scaffold and cloned into a mammalian expression plasmid driven by a constitutive polymerase II promoter. To screen these candidates for tau knockdown, plasmids were transfected into human osteosarcoma cells stably expressing the 1N4R isoform of MAPT mRNA with G272V and P301S mutations. After three days cells were lysed and tau protein was quantified by human tau ELISA. Ten artificial miRNA sequences were identified to significantly reduce tau protein expression by over 50% as compared to samples that had been transfected with an artificial miRNA control plasmid, denoted ‘Control’ (FIG. 1). For more details, please see general examples titled, “Plasmids, ITR vectors and AAV generation,” “(20) S cell culture and transfection,” and “Human tau (HT7 antibody) sandwich ELISA.”

### Example 2: Artificial miRNA Sequences Reduce Tau Expression in a Mouse Model of Tauopathy Following Intraparenchymal AAV Injection

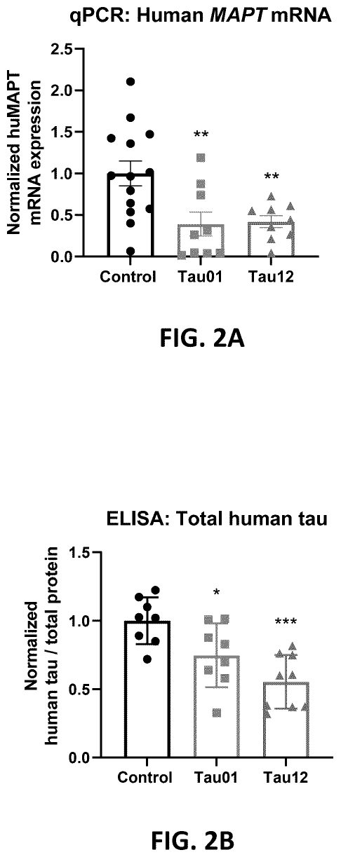

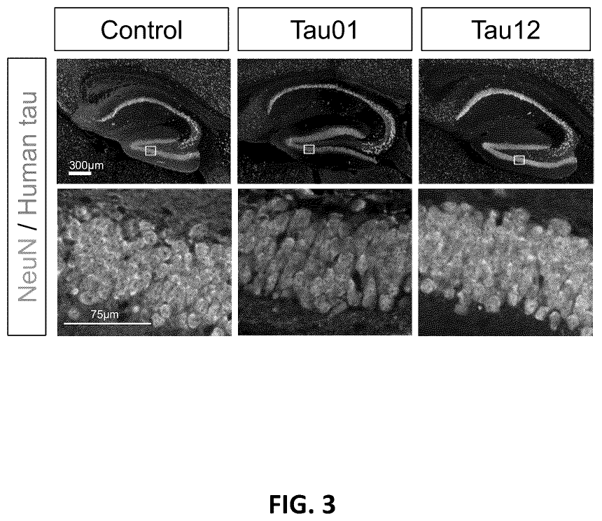

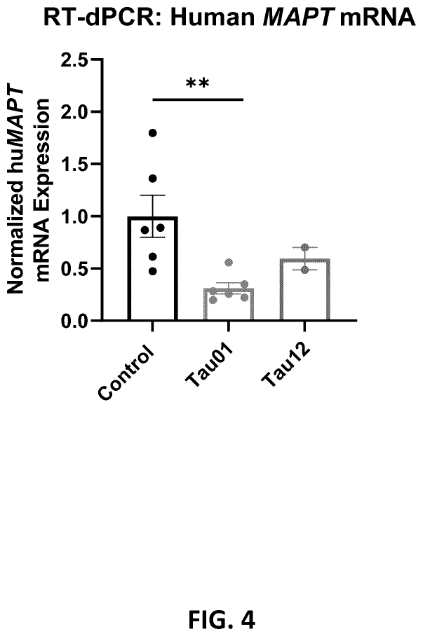

Two artificial miRNA sequences, Tau01 and Tau12 (see Table 1), were evaluated in the Tau22 mouse model. Tau22 animals overexpress the human 1N4R tau isoform with two mutations, G272V and P301S, under the neuron-specific Thy 1.2 promoter. These animals exhibit a progressive accumulation of phosphorylated tau isoforms and fibrillar aggregates, with associated neurodegeneration, gliosis, and behavioral deficits (Schindowski et al., 2006). AAVrh. 10-artificial miRNA vectors encoding our candidate artificial miRNAs were administered via intraparenchymal injection to the striatum of two-month-old Tau22 transgenic mice. After one month, human MAPT mRNA was significantly reduced by over 50% in the striatum of Tau01- and Tau12-treated animals, as measured by qPCR (FIG. 2A). Tau protein was measured by ELISA using the human tau-specific HT7 antibody. Tau01 and Tau12 significantly reduced tau protein expression by 25% and 45%, respectively (FIG. 2B). In a separate study, AAVrh. 10-artificial miRNA vectors were injected into the hippocampus of Tau22 mice, and hippocampal sections were immunostained for human tau after one month. Both Tau01 and Tau12 visibly reduced human tau expression in the dentate gyrus of the hippocampus, a region subject to progressive tau accumulation in the Tau22 mouse model (FIG. 3). The capsid AAV.SAN006 was also evaluated in this mouse model and it was found that intrastriatal administration of Tau01 significantly reduced human MAPT expression in the Tau22 striatum by over 50%. Tau12-treated animals showed a trend toward 40% knockdown (FIG. 4). For more details please see general examples titled “Plasmids, ITR vectors and AAV generation,” “Human tau (HT7 antibody) sandwich ELISA,” “Animal use and care,” “Stereotaxic injections with AAV-amiRNA vectors,” “Striatal tissue processing and MAPT mRNA quantification: by RT-qPCR,” “Striatal tissue homogenization following AAV.SAN006-amiRNA treatment,” “Striatal human MAPT mRNA quantification by RT-dPOR,” and “Hippocampal tau protein detection: Immunofluorescence.”

### Example 3: Artificial miRNA Sequences Show Accurate Strand Biasing and 5′ Processing in Vivo

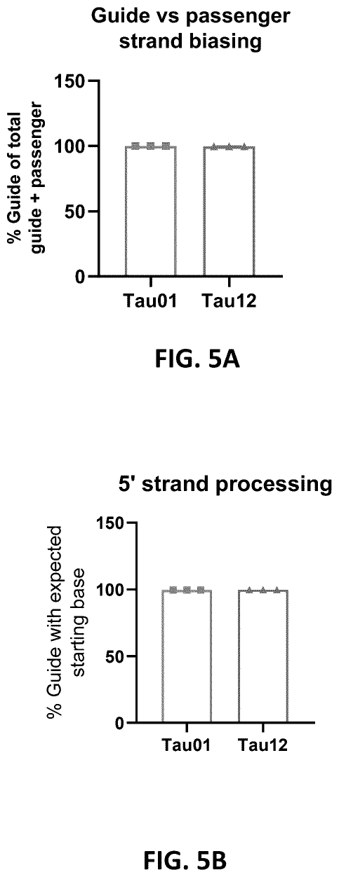

Each artificial miRNA is expressed as a pre-miRNA hairpin loop that is processed by the endogenous RNAi machinery to generate a mature 21 nucleotide duplex, the antisense guide strand of which is preferentially loaded into the RNA-induced silencing complex (RISC) to mediate target mRNA degradation while the passenger strand is excluded and degraded in the cytosol. Proper 5′ cleavage of the artificial miRNA guide sequence defines the seed sequence and is critical for on-target transcriptional silencing. Small RNA sequencing was used to evaluate guide vs passenger strand processing in vivo according to metrics known to contribute to on- and off-target activity of the artificial miRNA. Tau22 animals were administered intrastriatal injections of AAV-rh. 10-artificial miRNA vectors as described above, and total striatal RNA was isolated after four weeks post-injection. Small RNA libraries were prepared and sequenced to quantify guide and passenger strand expression levels and fidelity of strand cleavage at the 5′ and 3′ ends. For both Tau01 and Tau12, guide strands containing the antisense seed sequence targeting MAPT were highly enriched over their respective passenger strands, constituting an average of over 99% of the total artificial miRNA sequence expressed from the AAV-rh. 10 vector (FIG. 5A). Additionally, 5′ processing of the guide strand was over 99% accurate for each artificial miRNA (FIG. 5B). These results suggest that strand biasing for Tau01 and Tau12 is highly accurate and 5′ cleavage of the artificial miRNA sequences preserves the MAPT-targeting seed sequence as designed. For more details, please see general examples titled, “Plasmids, ITR vectors and AAV generation,” “Animal use and care,” “Stereotaxic injections with AAV-amiRNA vectors,” and “Small RNA sequencing.”

### Example 4: Artificial miRNA Sequences Reduce Tau Expression in a Mouse Model of Tauopathy Following Intravenous AAV-PHP.eB Injection

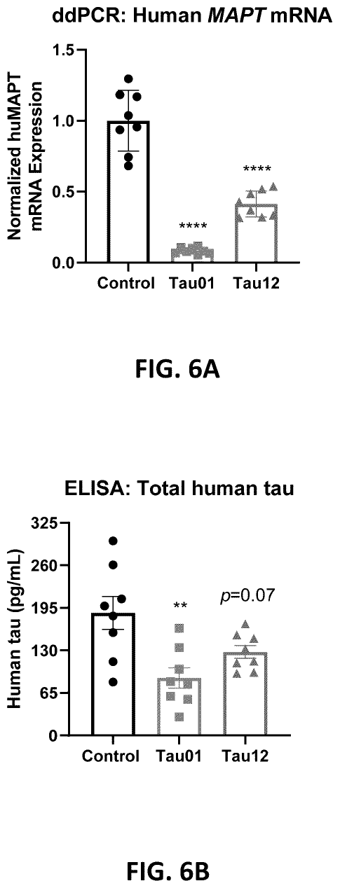

Clinical translation of tau-lowering therapies will require broad CNS transduction with target reduction following modest expression of the artificial miRNA. The AAV serotype PHP.eB can cross the blood brain barrier to yield widespread CNS transduction of C57/B16 mice following intravenous injection (Chan et al., 2017). AAV-PHP.eB vectors encoding artificial miRNAs were injected into the retroorbital sinus of Tau22 mice to evaluate tau reduction in hindbrain—a region severely affected in tauopathies including progressive supranuclear palsy. After one month, hindbrain tissue containing the cerebellum and brainstem was analyzed for MAPT knockdown, both at the mRNA and protein level. Both Tau01 and Tau12 significantly reduced human MAPT mRNA expression by 92% and 59%, respectively, as measured by ddPCR (FIG. 6A). Tau01 and Tau12 reduced human tau protein in hindbrain by 53% and 32%, respectively, as measured by ELISA (FIG. 6B). For more details, please see general examples titled, “Plasmids, ITR vectors and AAV generation,” “Animal use and care,” “Retroorbital injections with AAV-PHP.eB-amiRNA vectors,” “Hindbrain tissue homogenization,” “Hindbrain human MAPT mRNA quantification by ddP (′R,” and “Hindbrain human tau protein quantification by ELISA.”

### Example 5: Artificial miRNA Sequences Reduce Phospho Tau Pathology in a Late-Intervention Efficacy Paradigm

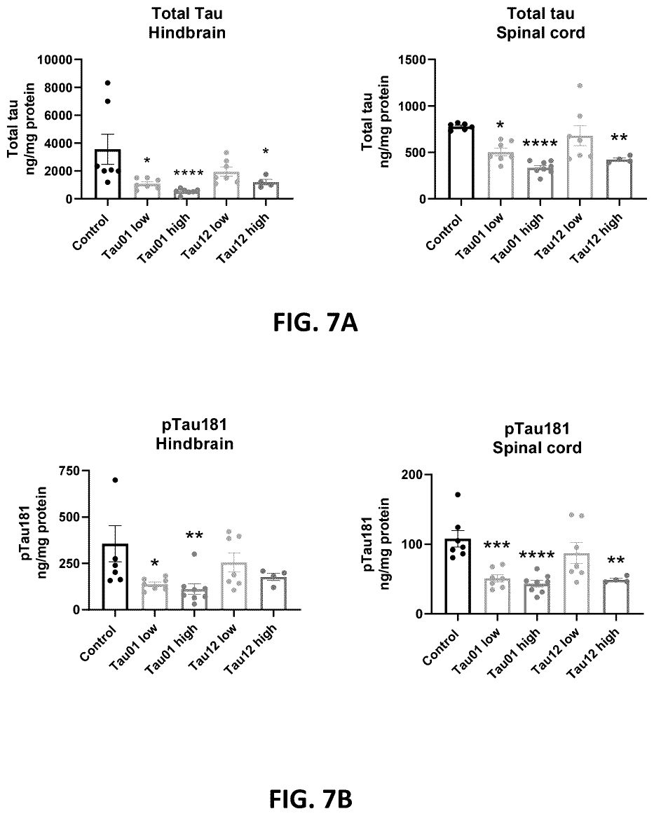

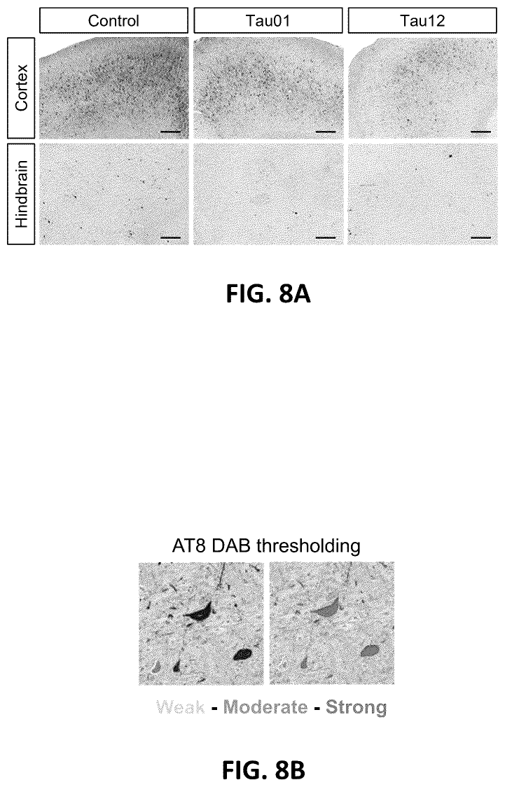

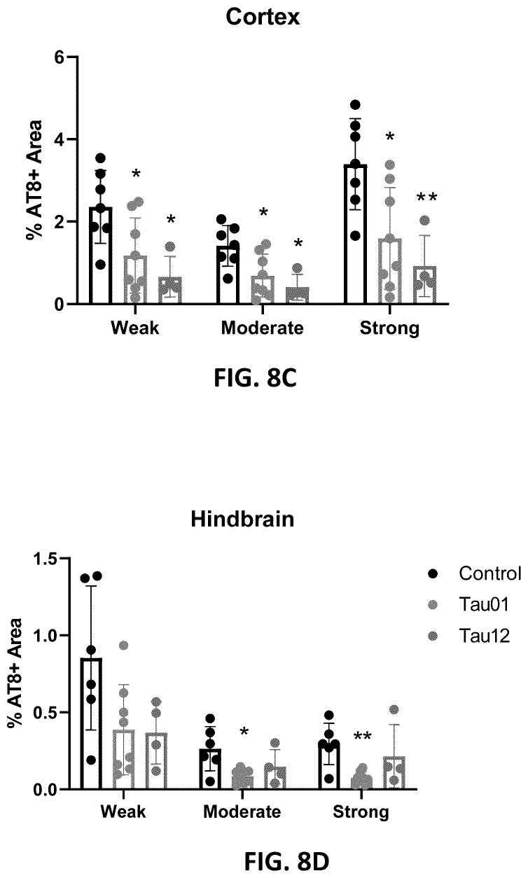

The progressive accumulation of phosphorylated tau (pTau) aggregates is a defining characteristic of tauopathies that is recapitulated in the Tau22 disease model. To further evaluate the efficacy of the Tau01 and Tau12 artificial miRNAs, AAV-PHP.eB vectors were administered by IV injection into Tau22 mice at six months of age, a point at which this model generates significant pTau aggregates. At three months post-injection, tissue homogenates from hindbrain and spinal cord were analyzed by Quanterix Simoa assay to measure total human tau protein and the tau phosphorylated at the 181 epitope (pTau181). Both low and high dose AAV-PHP.eB-Tau01 significantly reduced total tau and pTau181 in hindbrain and spinal cord, with greater than 50% reduction of both analytes in each tissue observed in the high dose Tau01 group. Significant reduction of total tau was achieved with high dose AAV-PHP.eB-Tau12 in both hindbrain and spinal cord, and significant pTau181 reduction was seen in the spinal cord (FIG. 7A-B). Tau pathology was also assessed in situ by immunohistochemistry, using the pTau antibody AT8 on tissue from animals in the high dose groups. In control animals, characteristic staining patterns representing tau neuropil threads and neurofibrillary tangles were observed in both the cortex and hindbrain (FIG. 8A). To quantify varying levels of pTau pathology, pixel-based detection was used to set low, moderate, and high AT8 intensity thresholds in each region of interest (FIG. 8B). In the cortex, significant reduction of AT8 positive area was observed at the low, medium, and high thresholds with both Tau01 and Tau12 treatments (FIG. 8C). In the hindbrain, significant reduction of the AT8+ area at moderate and high thresholds was observed in the high dose Tau01 group compared to control animals (FIG. 8D). For more details, please see general examples titled, “Plasmids, ITR vectors and AAV generation,” “Animal use and care,” “Retroorbital injections with AAV-PHP.eB-amiRNA vectors,” “Hindbrain tissue homogenization,” and “pTau Immunohistochemistry,” “Quanterix Simoa assays.”

### Example 6: Artificial miRNA Sequences Reduce Neurodegeneration Biomarker Neurofilament Light Chain (NfL) in a Late-Intervention Efficacy Paradigm

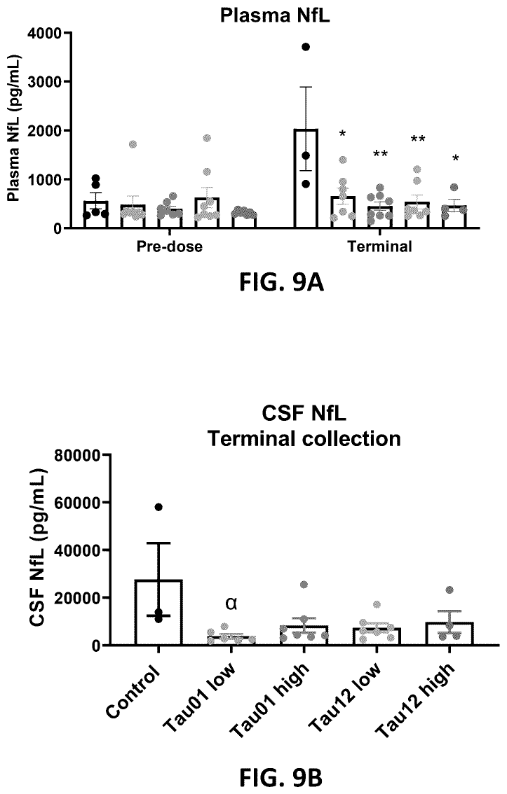

The Tau22 mouse model shows a progressive increase in plasma and cerebrospinal fluid (CSF) neurofilament light chain (NfL), a neurodegenerative biomarker that correlates with disease progression in patients with primary tauopathies (Brureau, 2017; Rojas et al., 2018). To determine whether artificial miRNA-mediated tau reduction also impacts neurodegeneration, a Quanterix Simoa assay was used to measure NfL in CSF and plasma. In control animals, an increase in plasma NfL was detected between the prestudy (FIG. 9A-left) at 6 months of age and terminal (FIG. 9A-right) 9 months of age timepoints. Across doses, a significant reduction in NfL was observed compared to control in plasma with both Tau01 and Tau12. A significant 86% reduction in CSF NfL was also observed at the terminal timepoint with low dose Tau01 versus controls (FIG. 9B). For more details, please see general examples titled, “Plasmids, ITR vectors and AAV generation,” “Animal use and care,” “Retroorbital injections with AAV-PHP.eB-amiRNA vectors,” and “Quanterix Simoa assays.”

### Example 7: AAV-RNAi Mediated Reduction of MAPT for the Treatment of Progressive Supranuclear Palsy

### Methods

In this example, in silico design tools were used to identify artificial miRNA (amiRNA) sequences with low off-targeting potential and homology across species. Sequences were tested for MAPT knockdown efficiency in vitro using U2OS cells stably expressing 4R human tau, with tau protein quantified by ELISA three days post-transfection. Top candidates were cloned into AAV vectors to evaluate MAPT knockdown and efficacy readouts in the Tau22 mouse model of tauopathy. These mice overexpress 1N4R human tau with two FTD-associated mutations that drive progressive neuronal accumulation of tau aggregates and subsequent neurodegeneration.

### Results

Several amiRNA candidates significantly reduced human tau protein expression in vitro. After three months of expression in the Tau22 mouse, lead amiRNA Tau01 significantly reduced MAPT mRNA expression. Furthermore, Tau01 reduced levels of pathological phosphorylated tau in the cortex, hindbrain, and spinal cord. Levels of the axonal damage biomarker neurofilament light were also significantly reduced in cerebrospinal fluid and plasma, suggesting that total tau reduction via AAV-driven RNAi can significantly impact neurodegeneration.

### CONCLUSIONS

These results support the use of these AAV-amiRNA vectors for the sustained reduction of pathogenic human tau. With a well-credentialed therapeutic target and clear path toward lead identification, we aim to develop a transformative treatment for patients with devastating neurodegenerative tauopathies like PSP.

What is claimed is:

1. An artificial microRNA comprising a first stand and a second strand, wherein
(a) the first strand and second strand form a duplex;
(b) the first strand comprises a guide region comprising a nucleotide sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, or SEQ ID NO: 22; and
(c) the second strand comprises a non-guide region that comprises a nucleotide sequence that is partially complementary to the nucleotide sequence of the guide region.

2. The artificial microRNA of claim 1, wherein the guide region comprises the sequence of SEQ ID NO: 1.

3. The artificial microRNA of claim 1, wherein the guide region comprises the sequence of SEQ ID NO: 12.

4. The artificial microRNA of claim 1, wherein the guide region comprises
(a) the sequence of SEQ ID NO: 1 and the non-guide region comprises the sequence of SEQ ID NO: 23;
(b) the sequence of SEQ ID NO: 2 and the non-guide region comprises the sequence of SEQ ID NO: 24;
(c) the sequence of SEQ ID NO: 3 and the non-guide region comprises the sequence of SEQ ID NO: 25;
(d) the sequence of SEQ ID NO: 4 and the non-guide region comprises the sequence of SEQ ID NO: 26;
(e) the sequence of SEQ ID NO: 5 and the non-guide region comprises the sequence of SEQ ID NO: 27;
(f) the sequence of SEQ ID NO: 6 and the non-guide region comprises the sequence of SEQ ID NO:28;
(g) the sequence of SEQ ID NO: 7 and the non-guide region comprises the sequence of SEQ ID NO: 29;
(h) the sequence of SEQ ID NO: 8 and the non-guide region comprises the sequence of SEQ ID NO: 30;
(i) the sequence of SEQ ID NO: 9 and the non-guide region comprises the sequence of SEQ ID NO: 31;
(j) the sequence of SEQ ID NO: 10 and the non-guide region comprises the sequence of SEQ ID NO: 32;
(k) the sequence of SEQ ID NO: 11 and the non-guide region comprises the sequence of SEQ ID NO: 33;
(1) the sequence of SEQ ID NO: 12 and the non-guide region comprises the sequence of SEQ ID NO: 34;
(m) the sequence of SEQ ID NO: 13 and the non-guide region comprises the sequence of SEQ ID NO: 35;
(n) the sequence of SEQ ID NO: 14 and the non-guide region comprises the sequence of SEQ ID NO: 36;
(o) the sequence of SEQ ID NO: 15 and the non-guide region comprises the sequence of SEQ ID NO: 37;
(p) the sequence of SEQ ID NO: 16 and the non-guide region comprises the sequence of SEQ ID NO: 38;
(q) the sequence of SEQ ID NO: 17 and the non-guide region comprises the sequence of SEQ ID NO: 39;
(r) the sequence of SEQ ID NO: 18 and the non-guide region comprises the sequence of SEQ ID NO: 40;
(s) the sequence of SEQ ID NO: 19 and the non-guide region comprises the sequence of SEQ ID NO: 41;
(t) the sequence of SEQ ID NO: 20 and the non-guide region comprises the sequence of SEQ ID NO: 42;
(u) the sequence of SEQ ID NO: 21 and the non-guide region comprises the sequence of SEQ ID NO: 43; or
(v) the sequence of SEQ ID NO: 22 and the non-guide region comprises the sequence of SEQ ID NO: 44;

5. The artificial microRNA of claim 4, wherein the guide region comprises the sequence of SEQ ID NO: 1 and the non-guide region comprises the sequence of SEQ ID NO: 23.

6. The artificial microRNA of claim 4, wherein the guide region comprises the sequence of SEQ ID NO: 12 and the non-guide region comprises the sequence of SEQ ID NO: 34.

7. The artificial microRNA of any one of claims 1-6, wherein the artificial microRNA targets tau mRNA.

8. The artificial microRNA of claim 7, wherein binding of the guide region to the coding sequence of the tau mRNA reduces expression of the protein tau.

9. An expression construct comprising a nucleic acid encoding the artificial microRNA of any one of claims 1-8.

10. The expression construct of claim 9, wherein the nucleic acid encoding the microRNA is operably linked to a promoter.

11. The expression construct of claim 9 or claim 10, wherein the nucleic acid encoding the artificial microRNA is cloned into an miRNA scaffold, wherein transcription of the expression construct forms a stem-loop structure.

12. A vector comprising the expression construct of any one of claims 9-11.

13. The vector of claim 12, wherein the vector is a rAAV vector.

14. A viral particle comprising the vector of claim 12, wherein the viral particle is an AAV particle encapsidating the rAAV vector

15. The viral particle of claim 14, wherein the viral particle comprises a modified AAV9 capsid protein.

16. A method of treating or preventing a tauopathy in a patient in need thereof, comprising administering to the patient a composition comprising a microRNA comprising a guide strand that binds to a tau mRNA and a passenger strand, wherein the guide strand comprises a nucleotide sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, or SEQ ID NO: 22.

17. The method of claim 16, wherein the tauopathy is progressive supranuclear palsy (PSP).

18. The method of claim 17, wherein the tauopathy is Alzheimer's disease (AD).

19. A method of reducing tau expression in a patient suffering from a tauopathy, comprising administering to the patient a composition comprising a microRNA comprising a guide strand that binds to a tau mRNA and a passenger strand, wherein the guide strand comprises a nucleotide sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, or SEQ ID NO: 22.
